(19) United States
(12) Patent Application Publication
(10) Pub. No.: US 2024/0327870 A1
(43) Pub. Date: Oct. 3, 2024

(54) NUCLEIC ACID VECTOR COMPOSITIONS

(71) Applicant: SISAF LTD, Guildford (GB)

(72) Inventors: Roghieh Suzanne SAFFIE-SIEBERT, Guildford (GB); Michael WELSH, Guildford (GB); Nasrollah TORABI-POUR, Guildford (GB)

(73) Assignee: SISAF LTD, Guildford (GB)

(21) Appl. No.: 18/580,636

(22) PCT Filed: Jul. 22, 2022
(86) PCT No.: PCT/GB2022/051938

§ 371 Date: Jan. 19, 2024

(30) Foreign Application Priority Data
Jul. 23, 2021 (GB) 2110646.3

(51) Int. Cl. C12N 15/88; A61K 39/00; A61K 39/215; A61K 39/35

(52) U.S. Cl. CPC C12N 15/88; A61K 39/0008; A61K 39/0011; A61K 39/215; A61K 39/35; A61K 2039/53; A61K 2039/55555

(57) ABSTRACT

A nucleic acid vector composition comprising: one or more enzymes and/or one or more fragments thereof; a nucleic acid; one or more particles comprising hydrolysable silicon; and one or more lipids. Also related products, medical uses, and methods.

### FIELD OF INVENTION

The invention relates to nucleic acid vector compositions and related methods and products. Nucleic acid vector compositions are especially useful in, although not limited to, the field of pharmaceuticals. Compositions of the invention are especially useful for protecting nucleic acid from enzymatic degradation.

### BACKGROUND

There is a need for improvements in excipients for biologically active agents. The provision of suitable excipients is required for advances in biomedical research to be fully translated into effective, safe and cost-effective treatments.

As an illustrative example, nucleic acids such as RNA have been proposed as therapeutic agents. Nucleic acid delivery for therapy or other purposes is well-known, particularly for the treatment of diseases such as cystic fibrosis and certain cancers, and mRNA has been recently used in effective vaccines against SARS-CoV-2. The term “gene therapy” may be used to refer to the delivery into a cell of a gene or part of a gene to produce a therapeutic effect, such as producing a therapeutic effect by repairing, reconstructing or compensating for defective genetic material. More broadly, the term “nucleic acid delivery” may be used to refer to any introduction of nucleic acid material into target cells. As non-limiting examples, nucleic acid delivery includes mRNA vaccination and the production of commercially useful proteins in so-called cell factories.

Delivery systems for delivering nucleic acids to cells fall into three broad classes, namely:
(i) those that involve direct injection of naked nucleic acids,
(ii) those that make use of viruses or genetically modified viruses, and
(iii) those that make use of non-viral delivery agents.

Each has its advantages and disadvantages. Although viruses as delivery agents have the advantages of high efficiency and high cell selectivity, they have the disadvantages of toxicity, triggering of inflammatory responses, and are poorly suited to the delivery of large DNA fragments. Accordingly, an mRNA vaccine may advantageously comprise injectable naked mRNA or non-viral delivery systems, such as polyplex vectors or lipid nanoparticle vectors.

Unfortunately, lower transfection efficiencies have been noted with non-viral nucleic acid delivery systems. Non-viral nucleic acid delivery systems are based on the compaction of nucleic acid into nanometric particles by electrostatic interaction between the negatively charged phosphate backbone of the nucleic acid and charged polymers, typically cationic lipids and/or peptides (Erbacher, P. et al, Gene Therapy, 1999, 6, 138-145). The mechanism my which these species are introduced into cells is proposed to involve endocytosis of intact complexes, in which complexes formed between the nucleic acid and the lipid become attached to the surface of a cell, then enter the cell by endocytosis. The complex then remains localised within a vesicle or endosome for some time and the nucleic acid component is released into the cytoplasm.

The polymer components of a non-viral delivery system associate electrostatically to form a vector complex. The lipid component shields both the nucleic acid and, to a degree, any peptide component(s) from degradation, endosomal or otherwise: for example, the lipid component may form a lipid bilayer shell which encapsulates other components of the delivery system, including nucleic acid molecules. Cationic lipids for such a use were developed by Felgner in the late 1980s and reported in Proc. Natl. Acad. Sci. USA 84, 7413-7417, 1987; and in U.S. Pat. No. 5,264,618. Felgner developed the now commercially-available cationic liposome known by the trade mark “Lipofectin”. The “Lipofectin” liposome is a spherical vesicle having a lipid bilayer of the cationic lipid DOTMA (2,3-dioleyloxypropyl-1-trimethyl ammonium) and the neutral phospholipid lipid DOPE (phosphatidyl ethanolamine or 1,2-dioleoyl-sn-glycero-3-phosphoethanolamine) in a 1:1 ratio. Various other cationic liposome formulations have since been devised, most of which combine a synthetic cationic lipid and a neutral lipid. In addition to the DOTMA analogues, there may be mentioned complex alkylamine/alkylamides, cholesterol derivatives, such as DC-Chol, and synthetic derivatives of dipalmitol, phosphatidyl-ethanolamine, glutamate, imidazole and phosphonate. However, cationic vector systems vary enormously in their transfection efficiencies in the presence of serum, which clearly impacts on their potential uses for in vivo gene therapy and/or nucleic acid delivery.

A peptide component for use in such complexes typically has two functionalities: a “head group” containing a cell surface receptor-, for example integrin-, recognition sequence, and a “tail” that can bind nucleic acid non-covalently. A peptide component can be designed to be cell-type specific or cell-surface receptor specific. For example a degree of integrin-specificity can confer a degree of cell specificity to the complex. Specificity results from the targeting to the cell-surface receptors (for example, integrin receptors), and transfection efficiencies comparable to some adenoviral vectors can be achieved (Jenkins et al. Gene Therapy 7, 393-400, 2000).

The non-viral delivery of messenger RNA (mRNA) to cells as so far been particularly problematic and limited by the lack of an efficient vector. Attempts to deliver mRNA using known non-viral vehicles that have been used successfully for DNA or siRNA have resulted in sub-optimal levels of protein expression. Furthermore, known non-viral vehicles have poor storage stability when packaged with mRNA. Overcoming the lipid bilayer to deliver RNA into cells has remained a major obstacle for the widespread development of RNA therapeutics.

Therefore, there is a need for vectors that are specifically tailored to the delivery of mRNA, which deliver high levels of mRNA to cells and lead to good levels of protein expression. There is also a need for compositions tailored to the delivery of mRNA that have good stability upon storage, in particular mRNA delivery complexes that retain their structure and functionality upon storage at moderate temperatures. Similar considerations apply to the delivery of other nucleic acid therapeutic agents, for example plasmid-derived DNA, in that there are challenges in maintaining good storage stability, especially at moderate temperatures, such as temperatures of about −5° C. to about 25° C.

A number of mRNA vaccines against SARS-CoV-2, including the Pfizer and BioNTech vaccine BNT162b2 (“Comirnaty”), and the Moderna CX-024414 vaccine, require cold chain storage and transport. This limits accessibility to the vaccine for low-income countries and adds cost and logistical complexity in all markets. It would be advantageous if vaccines could be stored and transported at standard refrigerator temperature (about −5° C.) or room temperature (about 20° C.). It would also be of benefit if vaccines could tolerate higher temperatures (for example, 30, 40 or 50° C.) for storage, or at the very least in the short-term for transport and distribution purposes.

Maintaining nucleic acid (for example mRNA or DNA) stability in an injectable composition, for example a vaccine composition, by means of low temperatures, as well as bringing logistical challenges, also has the technical limitation that the nucleic acid must be defrosted before injection, and that after injection it must remain stable in the body for long enough to show sufficient biological activity. This may require stability to be maintained during translocation around the body and/or escape from the endosomal compartment. Stability in vivo must also be maintained for long enough for sufficient translation into protein to take place.

Nucleic acid (and especially mRNA) is vulnerable to degradation, particularly enzymatic degradation. Degradation may be slowed by low temperature and/or by lyophilisation of the nucleic acid (e.g. mRNA), but each of those options come with disadvantages. Short lengths of nucleic acid can be made in an entirely synthetic production environment which can be kept free of degradative enzymes. Such short length nucleic acids may be useful for certain therapeutic applications such as siRNA. Longer nucleic acids cannot be made cost effectively in entirely synthetic production environments and are therefore typically made in production environments which include biologically-sourced material. For example, mRNA may be made by in vitro transcription in which the transcription enzymes derive from biologically sourced material (by biologically sourced material is meant material, particularly nucleic acid, which is either produced in a cell-culture based system, or produced in vitro using synthetic enzymes, for example enzymes produced in a cell-culture based system). DNA (for example plasmid DNA or pDNA) may be extracted from a culture of cells. The use of biologically sourced material increases the chance that biologically-sourced degradative enzymes will be present, unintentionally, in the nucleic acid preparation. This may require extensive purification processes (which are costly and result in loss of yield) and it may not be possible or cost effective to completely remove all degradative enzyme from a biologically-sourced nucleic acid preparation, for example a preparation of in vitro transcribed (IVT) mRNA or a preparation of DNA such as pDNA. Moreover, during translocation around the body and/or escape from the endosomal compartment, nucleic acid is exposed to physiological and intracellular conditions, typically including contact with degradative enzymes.

Encapsulation with lipid or condensation with a peptide or cationic polymer (such as protamine) have been used in the prior art to protect nucleic acid (for example, mRNA for gene therapy or vaccination) from degradation. Condensation with a peptide or other polymer relies on the peptide or polymer remaining intact and retaining its charge. There is a need for improved excipients for increasing the stability of nucleic acid (for example mRNA) and for improving its resistance to enzymatic degradation, especially if resistance to enzymatic degradation can be achieved without having to freeze the preparation of nucleic acid. There is also a need for improved excipients for increasing the stability of multiple components of a composition comprising biologically sourced nucleic acid and lipid and/or peptide or other polymer.

### SUMMARY OF THE INVENTION

The present invention is based on an appreciation that hydrolysable silicon in the presence of a lipid can be used to stabilise nucleic acid such as mRNA or DNA (e.g. in vitro transcribed mRNA or plasmid DNA) by protecting the nucleic acid from enzymatic degradation.

This may include stabilisation of the nucleic acid in a pharmaceutical composition (e.g. a vaccine) during storage, for example prior to the pharmaceutical composition being administered to a subject in need thereof. It may include stabilisation during translocation of the nucleic acid around the body, and/or escape from the endosomal compartment (when nucleic acid is exposed to physiological conditions, typically including contact with degradative enzymes).

Thus, according to a first aspect of the invention, there is provided a nucleic acid vector composition comprising:
one or more enzymes and/or one or more fragments thereof;
a nucleic acid;
one or more particles comprising hydrolysable silicon; and
one or more lipids.

The one or more particles comprising hydrolysable silicon may remove or sequester water molecules, thereby preventing the one or more enzymes from degrading the nucleic acid, on the basis that enzymes require an aqueous environment in order for them to catalyse the degradation of a nucleic acid. Optionally, the one or more particles remove water molecules by reaction of silicon, or silicon-containing moieties, on the surface of the one or more particles, with water molecules. Optionally, the one or more particles sequester water molecules by trapping the water molecules in pores present on the surfaces of the one or more particles. Optionally, the one or more particles sequester water molecules in both of these ways. Thus, water molecules available to react with the nucleic acid in an enzyme-catalysed reaction may be reduced or eliminated. Overall, therefore, the one or more particles comprising hydrolysable silicon increase the stability of the nucleic acid, compared to a composition without the one or more particles.

The one or more enzymes may comprise one or more enzymes for forming an enzyme-substrate complex with a nucleic acid. In the composition of the first aspect of the invention, it may be that enzyme-substrate complexes are capable of forming, yet the steps required for an enzyme-catalysed reaction cannot occur, or can only occur at a reduced rate, because of the reduced availability of water molecules.

The one or more enzymes optionally comprise one or more nucleases. The one or more enzymes optionally comprise one or more polymerases: in certain embodiments, therefore, the one or more enzymes optionally comprise one or more RNA polymerases, which may be selected from one or more of T7, SP6, and T3 RNA polymerase. The one or more enzymes optionally comprise both one or more nucleases, and one or more polymerases.

As used herein, the term nuclease refers to an enzyme capable of cleaving phosphodiester bonds between nucleotides in a nucleic acid molecule. A nuclease may be capable of effecting single stranded breaks in a target molecule. A nuclease may be capable of effecting double stranded breaks in a target molecule. As used herein, the term exonuclease refers to an enzyme capable of digesting a nucleic acid molecule by cleaving nucleotides one at a time from the end (exo) of a polynucleotide chain. As used herein, the term endonuclease refers to an enzyme capable of digesting a nucleic acid molecule by cleaving nucleotides one at a time starting from the middle (endo) of a polynucleotide chain. A nuclease may be a deoxyribonuclease, also known as a DNase, which acts on DNA. A nuclease may be a ribonuclease, also known as an RNase, which acts on RNA.

In general terms, a polymerase is an enzyme which catalyses the formation of a polymer. Thus, the term DNA polymerase as used herein refers to an enzyme which catalyses the formation of a DNA polymer. RNA polymerase as used herein is an enzyme which catalyses the formation of an RNA polymer. A nucleic acid molecule has a sugar-phosphate backbone, which is typically exposed on the hydrophilic surface of the molecule and is thus a part of the molecule which is particularly vulnerable to hydrolysis. Nucleophilic cleavage of phosphodiester bonds in the backbone may occur, for example, via intermolecular reactions (with a nucleophile present in solution, e.g. H₂O) and/or intramolecular attack (such as by the 2′—OH group in RNAs, or other nucleophilic groups). Other modes of degradation, for example via oxidation of one or more nucleobases, and/or of one or more sugar moieties, are also conceivable.

In the nucleic acid vector composition according to the first aspect of the invention, the ratio of enzyme and/or fragment thereof to nucleic acid by weight is optionally in the range of 1:1×10¹² to 1:1, for example 1:1×10¹¹ to 1:1, 1:1×10¹⁰ to 1:1, 1:1×10⁹ to 1:1, 1:1×10⁷ to 1:1, 1:1×10⁶ to 1:1, 1:1×10⁵ to 1:1, 1:1×10⁴ to 1:1, 1:1000 to 1:1, or 1:100 to 1:1.

As used herein, the term enzyme activity refers to the rate of a reaction catalysed by the enzyme in question. The one or more enzymes, present in the composition of the first aspect of the invention, optionally have an activity, on a nucleic acid (e.g., mRNA) substrate, of at least 1 nmol min⁻¹ at a pH of 7.4 and a temperature of 25° C., for example at least 10 nmol min⁻¹, at least 100 nmol min⁻¹, at least 1 μmol min⁻¹, at least 2 μmol min⁻¹, at least 5 μmol min⁻¹, at least 10 μmol min⁻¹, or at least 50 μmol min⁻¹ under these conditions.

The one or more enzyme fragments may have an activity on a nucleic acid (e.g., mRNA) substrate, at a pH of 7.4 and a temperature of 25° C., of at least 20%, at least 30%, at least 40%, at least 50%, at least 60%, at least 70%, and particularly, at least 80%, of the activity of a corresponding whole enzyme molecule under the same pH and temperature conditions. Accordingly, the one or more enzyme fragments may retain one or more intact active sites of a corresponding whole enzyme molecule. The one or more enzyme fragments may retain one or more intact secondary structures, for example one or more intact beta sheets and/or alpha helices, of a corresponding whole enzyme molecule.

Optionally, the nucleic acid vector composition is in the form of a lipid nanoparticle, optionally having a core encapsulated by a shell, wherein the shell comprises one or more lipids, for example in a lipid bilayer, and the core comprises the nucleic acid and the one or more particles comprising hydrolysable silicon.

Without wishing to be bound by theory, it is thought in a conventional lipid nanoparticle, formulated with one or more lipids and with a nucleic acid (but without hydrolysable silicon) typically has a core encapsulated by a shell. The shell is often a lipid bilayer (for example, comprising DSPC, PEG₂₀₀₀, and the like). The core is typically amorphous, is encapsulated by the shell, and comprises nucleic acid, water, and other components such as cationic lipid, and/or cholesterol, depending on the formulation employed. Although this may protect the nucleic acid (e.g. mRNA) from an external medium, nucleic acid molecules in the core may nonetheless be in contact with water molecules. For example, it has been suggested that water pores surrounded by inverted cationic lipids are present in the core (Viger-Gravel et al., J. Phys. Chem. B 122 (7), 2073-2081 (2018)). It has been suggested that the core may have a water content of from 10 to 40% by volume, such as 20 to 30% by volume, for example 23 to 25% by volume. The mRNA may be located inside water cylinders in a disordered inverse hexagonal phase, as reported by Areta et al., Proc. Natl. Acad. Sci., 115 (15), E3351-E3360 (2018).

Thus, the composition of the present invention may have some similarities to a conventional lipid nanoparticle, in that it may be in the form of a lipid nanoparticle, optionally having a core encapsulated by a shell, wherein the shell comprises one or more lipids, for example in a lipid bilayer, and the core, which optionally is amorphous, comprises the nucleic acid and the one or more particles comprising hydrolysable silicon. However, in contrast to conventional lipid nanoparticles, in the composition according to the first aspect of the present invention the particles comprising hydrolysable silicon may sequester water molecules away from the nucleic acid, thereby retarding or preventing the reaction of the nucleic acid with water in an enzyme-catalysed reaction, for example, reaction of the sugar-phosphate backbone with water: so as to stabilise the nucleic acid in the lipid nanoparticle.

Optionally, components of the delivery system, such as lipid and/or peptide molecules, may bind to one or more of the silicon particles, resulting in a stabilised complex in which both the hydrolysable silicon and other components of the delivery system, such as lipids and/or peptides, are stabilised. Positively charged species such as cationic lipids (and other lipid components bearing a positive charge, including, but not limited to, phospholipids) may bind to one or more particles comprising hydrolysable silicon, thereby stabilising these positively charged components.

Thus, preferably, degradation of the nucleic acid (for example mRNA or pDNA) at room temperature (20° C.) is reduced by at least half, more preferably by a factor of at least 5, 10, 35, 50, 100, 500 or 1000 compared to an equivalent composition without the silicon particles.

The nucleic acid may be or comprise RNA, such as mRNA. The nucleic acid may be or comprise DNA. Where the nucleic acid is or comprises mRNA, the mRNA may be in vitro transcribed mRNA. Where the nucleic acid is or comprises DNA, the DNA may be plasmid DNA. Where the nucleic acid is or comprises mRNA, the mRNA may comprise a protein-encoding open reading frame and optionally one or more of:
a 5′ cap;
a poly (A) tail; and
one or more untranslated regions.

Optionally, the open reading frame of the mRNA encodes an antigen of a pathogen. Optionally, the open reading frame of the mRNA encodes a spike protein antigen of SARS-CoV-2.

Optionally, in certain preferred embodiments of all aspects of the invention, the open reading frame of the mRNA encodes a tumour-specific antigen. As used herein, the term tumour-specific antigen may refer to an antigen that arises, in one or more malignant cancer cells, from non-synonymous somatic mutation (leading to a neoantigen) or viral-integrated mutation (leading to an oncoviral antigen). Tumor-specific antigens may thus refer to antigens that are completely absent from (not expressed by) non-cancerous (healthy, normal) cells.

Optionally, the open reading frame of the mRNA encodes a tumour-associated antigen. As used herein, the term tumour-associated antigen may refer to an antigen that is over-expressed in a malignant cancer cell, compared to a non-cancerous (healthy, normal) cell, for example due to genetic amplification or post-translational modifications. The term tumour-associated antigen may encompass overexpressed antigens (which term may refer to proteins that are moderately expressed in non-cancerous (healthy, normal) cells, but expressed abundantly in malignant cancer cells); differentiation antigens (which term may refer to proteins that are selectively expressed by the cell lineage from which the malignant cells evolved, an example being prostate-specific antigen); and cancer-germline antigens (which term may refer to antigens that are normally limited to reproductive tissues, but which are aberrantly expressed in a malignant cancer cell: for example, melanoma antigen family A3 (MAGE-A3); New York Esophageal Squamous Cell Carcinoma-1 Antigen (NY-ESO-1); and Preferentially Expressed Antigen in Melanoma (PRAME))

When the open reading frame of the mRNA encodes a cancer-associated antigen or a cancer-specific antigen, the nucleic acid vector composition may be suitable for use in a prophylactic or therapeutic vaccine composition.

Optionally, the open reading frame of the mRNA encodes an allergen (including but not limited to one or more nut allegens: which in turn include, but are not limited to: one or more seed storage proteins, such as vicilins, legumins, albumins: one or more plant defense related proteins; and one or more profilins).

Optionally, the open reading frame of the mRNA encodes a protein that modulates an immune, autoimmune, or inflammatory disease (including, but not limited to, lupus, atherosclerosis, chronic obstructive pulmonary disease, inflammatory bowel disease, multiple sclerosis, psoriasis, a rheumatic disease, uveitis, atopic dermatitis, and pulmonary fibrosis).

Optionally, the nucleic acid vector composition according to the first aspect of the invention further comprises an amino acid, such as glycine. Additionally or alternatively, the composition further comprises one or more disaccharides, such as trehalose.

According to an alternative aspect of the invention, there is provided a nucleic acid vector composition comprising:
one or more enzymes;
a nucleic acid;
one or more particles comprising hydrolysable silicon; and
one or more lipids.

According to a second aspect of the invention, there is provided a method of preparing the nucleic acid vector composition according to the first aspect of the invention, the method comprising:
obtaining nucleic acid by in vitro transcription from a DNA template or purification from a biological source; and
combining the nucleic acid with one or more particles comprising hydrolysable silicon and one or more lipids.

According to a third aspect of the invention, there is provided a pharmaceutical composition comprising the nucleic acid vector composition of the invention, wherein the pharmaceutical composition is a vaccine composition.

Preferably, the nucleic acid (for example mRNA or pDNA) in a pharmaceutical composition according to the third aspect of the invention, comprising the nucleic acid vector composition of the first aspect of the invention, has a half-life at 4° C. of at least 3 months, at least 6 months or at least 12 months.

According to a fourth aspect of the invention, there is provided a pharmaceutical composition according to the third aspect of the invention, for use as a medicament.

According to a fifth aspect of the invention, there is provided a method of treating or preventing a disease or disorder, comprising: administering to a subject in need thereof a pharmaceutical composition according to the third aspect of the invention.

According to a sixth aspect of the invention, there is provided a method of providing a vaccination to a subject, comprising subcutaneous or intramuscular administration of a pharmaceutical composition according to the third aspect of the invention (when it is in a form suitable for intramuscular injection).

According to a seventh aspect of the invention, there is provided the use of a pharmaceutical composition according to the third aspect of the invention as a lipopolyplex transfection vector.

According to an eighth aspect of the invention, there is provided the use of a pharmaceutical composition according to the third aspect of the invention in the manufacture of a medicament.

### DESCRIPTION OF THE DRAWINGS

FIG. 1 compares the Zeta potentials of siRNA loaded and unloaded compositions. Each formulation is labelled F1 to F5;

FIG. 2 shows a comparison of Zeta potentials of siRNA loaded (columns with lighter dotted fill) and unloaded (columns with darker hashed fill) compositions, wherein the silicon nanoparticle is surface treated with stearylamine (FIG. 2); PC (FIG. 3); or lecithin (FIG. 4), and the amine is arginine:

FIG. 5 shows the result of an experiment to determine the transfection efficiency of compositions in HCES cells. Cells of the first column are stained with DAPI and fluoresce (in the original) in blue, showing the nucleus. Cells of the second column fluoresce in green (in the original) indicating successful transfection with formulations F2 to F5 of the invention;

FIG. 6 shows the result of a gel electrophoresis experiment, indicating that silicon nanoparticles prepared according to the invention successfully entrap mRNA, particularly at ratios of silicon nanoparticles to mRNA of 2:1 or higher;

FIG. 7 shows the result of a spectrophotometry experiment, confirming that silicon nanoparticles prepared according to the invention successfully entrap mRNA, particularly at ratios of silicon nanoparticles to mRNA of 2:1 or higher;

FIG. 8 shows the result of an experiment to determine the transfection efficiency of a silicon nanoparticle delivery system loaded with siRNA;

FIG. 9 shows the result of an experiment to determine the post-transfection viability of cells treated with a silicon nanoparticle delivery system loaded with siRNA;

FIG. 10 shows the result of an experiment to determine the extent of siRNA-induced gene silencing when siRNA is delivered to cells using silicon nanoparticle delivery systems;

FIG. 11 shows the result of an experiment evaluating in vivo ocular siRNA delivery by topical silicon nanoparticle formulations;

FIGS. 12 and 13 show the result of an experiment to determine murine corneal luciferase expression in vivo by live animal imaging, during treatment with a silicon nanoparticle delivery system loaded with siRNA; and

FIG. 14 shows the result of an experiment to determine the nucleic acid binding efficiency for hsDNA for Si nanoparticle-containing formulations and for liposome formulations without Si nanoparticles.

FIG. 15 shows the appearance of silicon wafer before and after milling into powder.

FIG. 16 shows an SEM image of hand-milled silicon particles which was used to assess their size.

FIG. 17 shows a TEM image of the a hand milled silicon particles

FIG. 18 shows a TEM image of the silicon particle of FIG. 17 after complexation with components of the invention including RNA.

FIG. 19 shows a TEM image of a silicon particle after complexation with components of the invention including RNA. It is similar to the particle shown in FIG. 18 except it is made with boron-doped silicon.

FIGS. 20 and 21 show nucleic acid binding efficiency (respectively, mRNA or hsDNA) as determined for a “Biocourier” silicon particle containing formulation of the invention (solid bars) and a corresponding “liposome” formulations (open bars) with the absence of the silicon particle.

FIG. 22 shows the results of a gel retardation assay of (A) mRNA-SIS0012 incubated with bovine serum (BS) for different durations of time and (B) mRNA extracted from mRNA-SIS0012 before and after incubation with bovine serum. Naked mRNA in nuclease free water was used as a control. DNA ladder was used as a size guide.

FIG. 23 shows the results of a gel retardation assay of (A) mRNA-SIS0013 incubated with bovine serum (BS) for different durations of time and (B) mRNA extracted from mRNA-SIS0013 before and after incubation with bovine serum. Naked mRNA in nuclease free water was used as a control. DNA ladder was used as a size guide.

FIG. 24 shows the results of a gel retardation assay of (A) mRNA-LNP formulated without silicon and incubated with bovine serum (BS) for different durations of time and (B) mRNA extracted from the LNP before and after incubation with bovine serum. Naked mRNA in nuclease free water was used as a control. DNA ladder was used as a size guide.

FIG. 25 shows the results of a gel retardation assay of naked mRNA, incubated with bovine serum (BS) for different durations of time. Naked mRNA in nuclease free water was used as a control. DNA ladder was used as a size guide.

### DETAILED DESCRIPTION

### Biologically Sourced Nucleic Acid

Optionally the invention, according to certain embodiments, relates to biologically sourced nucleic acid. This means nucleic acid either produced in a cell-culture based system, or nucleic acid produced in vitro using enzymes produced in a cell-culture based system. Biologically sourced nucleic acids may include RNA (such as mRNA) and DNA (such as pDNA) extracted from cellular material or transcribed in vitro (including where there is subsequent chemical modification). It does not include nucleic acids made by a wholly chemical synthetic route such as phosphoramidite chemical synthesis.

In Vitro Transcribed mRNA

In certain preferred embodiments of all aspects of the invention the nucleic acid vector composition of the invention comprises a preparation of in vitro transcribed (IVT) mRNA.

The term “mRNA” is used herein to refer to messenger RNA. The term encompasses unmodified mRNA and modified mRNA. In this connection, it will be appreciated that mRNA according to certain embodiments of all aspects of the invention can be chemically modified to enhance its therapeutic properties, such as enhanced activity, increased serum stability, reduced off-targeting and lower immunological activation.

Chemical modifications to the RNA may include any modifications commonly known in the art, for example: modification with labels which are known in the art; methylation; caps such as 5′ caps (e.g., Cap 0 which requires a 7-methylguanosine connected by a triphosphate bridge to the first nucleotide; Cap 1 which requires methylation of the 2′-hydroxyl group of the first cap-proximal nucleotide; or Cap 2 having an additional 2′-O-methylation of the second nucleotide; or a modified Cap 0, Cap 1 or Cap 2 structure, such as the modified Cap 1 structure m⁷G⁺m^{3′}-5′-ppp-5′-Am); poly (A) tails; substitution of one or more of the naturally occurring nucleosides with an analogue, for example substitution of uridine with N1-methyl-pseudouridine; and modification of the inter-nucleoside linkages.

In some embodiments, the mRNA may be at least 100, at least 200, at least 300, at least 500, or at least 1000 or 2000 base pairs in length.

Plasmid DNA (pDNA)

In other embodiments, the nucleic acid vector composition comprises a preparation of DNA. Preferably this is plasmid DNA which includes any DNA in the form of a plasmid of which has been obtained from a plasmid (for example by being cut out of a plasmid).

In Vitro mRNA Transcription

In certain preferred embodiments, the method of the second aspect of the invention of preparing the nucleic acid vector composition requires synthesising mRNA by in vitro transcription (IVT) from a linear DNA template.

The linear DNA template may be produced in advance. For example, in some embodiments, a DNA template may be produced in advance by linearisation of a purified plasmid, or by amplification of the region of interest using PCR.

Transcription can be carried out using RNA polymerase. For example, transcription can in some embodiments be carried out using T7, SP6 or T3 RNA polymerase. In certain preferred embodiments, for example when the open reading frame of the mRNA encodes a spike protein antigen of SARS-CoV-2, T7 RNA polymerase can be used.

Reactants used in transcription can therefore include: the linear DNA template; RNA polymerase (such as T7, SP6 or T3 RNA polymerase); and nucleoside triphosphate substrates. The nucleoside triphosphate substrates may include modified nucleoside triphosphate substrates, for example N1-methyl-pseudouridine. In some embodiments, polymerase cofactor MgCl₂ may be included. In some embodiments, a pH buffer solution (for example a pH buffer solution containing polyamine and antioxidants) may be included.

5′ capping of the mRNA can optionally be performed during the IVT reaction, i.e., 5′ capping can be co-transcriptional. This can be achieved by substituting a part of the guanosine triphosphate substrate for a cap analog. Optionally, co-transcriptional capping can be performed using a CleanCap™ reagent.

As an alternative to co-transcriptional capping, mRNA can in some embodiments be capped in a second reaction (after transcription) which is catalysed by the vaccinia capping enzyme (VCC) together with a methyl donor. In certain preferred embodiments, for example when the open reading frame of the mRNA encodes a spike protein antigen of SARS-CoV-2, 5′ capping is carried out using vaccinia capping enzyme and Vaccinia 2′ O-methyltransferase.

A 5′ cap may in some embodiments be selected from Cap 0, Cap 1, or Cap 2, or a modified Cap 0, modified Cap 1 (for example, a m⁷G⁺m^{3′}-5′-ppp-5′-Am cap) or a modified Cap 2.

mRNA and pDNA Purification

Preparing the nucleic acid vector composition of the invention requires purifying the nucleic acid (for example mRNA or pDNA). In embodiments where the nucleic acid is mRNA, this may require purifying the mRNA from the transcription reaction mixture (which contains impurities, including, in some embodiments, one or more of: enzymes, residual nucleoside triphosphates, DNA template, and aberrant mRNAs formed during IVT) to form a preparation of in vitro transcribed mRNA.

Optionally, purification can include, but is not limited to, one or more of: size exclusion chromatography (SEC); ion pair reverse-phase chromatography (IPC); ion exchange chromatography (IEC); affinity based separation; tangential flow filtration (TFF); core bead chromatography; hydroxyapatite chromatography; mRNA precipitation combined with TFF (during TFF, the membrane captures the precipitated mRNA product while other impurities are removed by diafiltration); DNA template removal by performing a digestion with immobilised DNase; and the use of tagged DNA template that can be removed after IVT using affinity chromatography. In certain preferred embodiments, for example when the open reading frame of the mRNA encodes a spike protein antigen of SARS-CoV-2, purification can involve oligo-dT affinity purification, buffer exchange by tangential flow filtration into sodium acetate (pH 5.0) and sterile filtration.

It will be understood that even when purification is complete, the preparation of in vitro transcribed mRNA may still comprise, in at least trace amounts, one or more of: linear DNA; one or more RNA polymerases; and one or more nucleoside triphosphates. For example, the preparation of in vitro transcribed mRNA may comprise at least 0.01% v/v linear DNA. The preparation of in vitro transcribed mRNA may comprise at least 0.01% v/v RNA polymerases. the preparation of in vitro transcribed mRNA may comprise at least 0.01% v/v nucleoside triphosphates.

pDNA may be purified from a cell-derived (including microbial cell-derived, such as bacterial or yeast cell-derived) extract by analogous methods.

### Preparing the Nucleic Acid Vector Composition

In the method of preparing a nucleic acid vector composition according to the second aspect of the invention, the preparation of nucleic acid (for example pDNA or in vitro transcribed mRNA) is then combined with water, a particle comprising hydrolysable silicon, and one or more lipids. The composition may optionally further comprise one of more amino acid (for example glycine or a mixture of nucleic acids including glycine) and optionally one or more non-reducing disaccharide such as trehalose. A suitable preparation method may include dispersing the lipid component in a solvent such as methanol; generating a thin film of lipid by evaporating the solvent, for example in rotary evaporator; hydrating the lipid with an aqueous solution containing activated hydrolysable silicon particles, for example particles having an average particle size less than 100 nm, a non-reducing disaccharide such as trehalose and one or more amino acid such as glycine. The composition may optionally be passed though filters, for example 0.4 and 0.1 um filters to achieve complexation and dispersal of the particles. The composition may be optionally be stored at 4° C. if required to allow further complexation to take place. A carrier prepared thus may then be complexed with aqueous solution of nucleic acid (such as plasmid DNA or mRNA at ratios ranging from 1:6 to 1:16, where 1 represents the nucleic acid). Preferred ratios are 1:8-1:12, where 1:8 usually allows a small excess of biological, and 1:12 allows a small excess of the carrier.

mRNA Structure

In some embodiments of all aspects of the invention where the nucleic acid is mRNA, a molecule of mRNA can optionally comprise, in addition to a protein-encoding open reading frame, one or more of: a 5′ cap: a poly (A) tail; and one or more untranslated regions.

In some embodiments, the protein-encoding open reading frame of the mRNA encodes an antigen, thereby providing a formulation which is a vaccine. The antigen may be a viral antigen, for example an antigen of SARS-CoV-2, for example an antigen which is or which derives from the spike protein of SARS-CoV-2 or a part thereof. Thus, the open reading frame of the mRNA may in some embodiments encode a spike protein antigen of SARS-CoV-2. In some embodiments, the open reading frame can encode a mutated version of a naturally occurring protein. For example, in some embodiments when the open reading frame encodes the spike protein of SARS-CoV-2 or a part thereof, two mutations can be included in which the original amino acids are replaced with prolines. Without wishing to be bound by theory, it is thought that this can ensure the resultant S glycoprotein remains in an antigenically optimal pre-fusion conformation.

In some embodiments, the protein-encoding open reading frame of the mRNA may encode multiple proteins. For example, the protein-encoding open reading frame of the mRNA may encode a viral antigen and an adjuvanting protein, or multiple viral antigens. In certain preferred embodiments, for example when the open reading frame of the mRNA encodes a spike protein antigen of SARS-CoV-2, the open reading frame may additionally encode a signal peptide, for example a S glycoprotein signal peptide.

5′ cap

As described above, the mRNA can be modified by having a 5′ cap. The 5′ cap may have the Cap 0 (m⁷GpppN) structure, which requires a 7-methylguanosine connected by a triphosphate bridge to the first nucleotide. The 5′ cap may have the Cap 1 (m⁷GpppNm) structure, which requires methylation of the 2′-hydroxyl group of the first cap-proximal nucleotide. The 5′ cap may have the Cap 2 (m⁷GpppNm pN^{m}) structure, having an additional 2′-O-methylation of the second nucleotide. The cap may have a modified Cap 0, modified Cap 1, or modified Cap 2 structure. In certain preferred embodiments, for example when the open reading frame of the mRNA encodes a spike protein antigen of SARS-CoV-2, the 5′ cap is Cap 1, or the 5′ cap is a modified Cap 1 having the m⁷G⁺m^{3′}-5′-ppp-5′-Am cap structure.

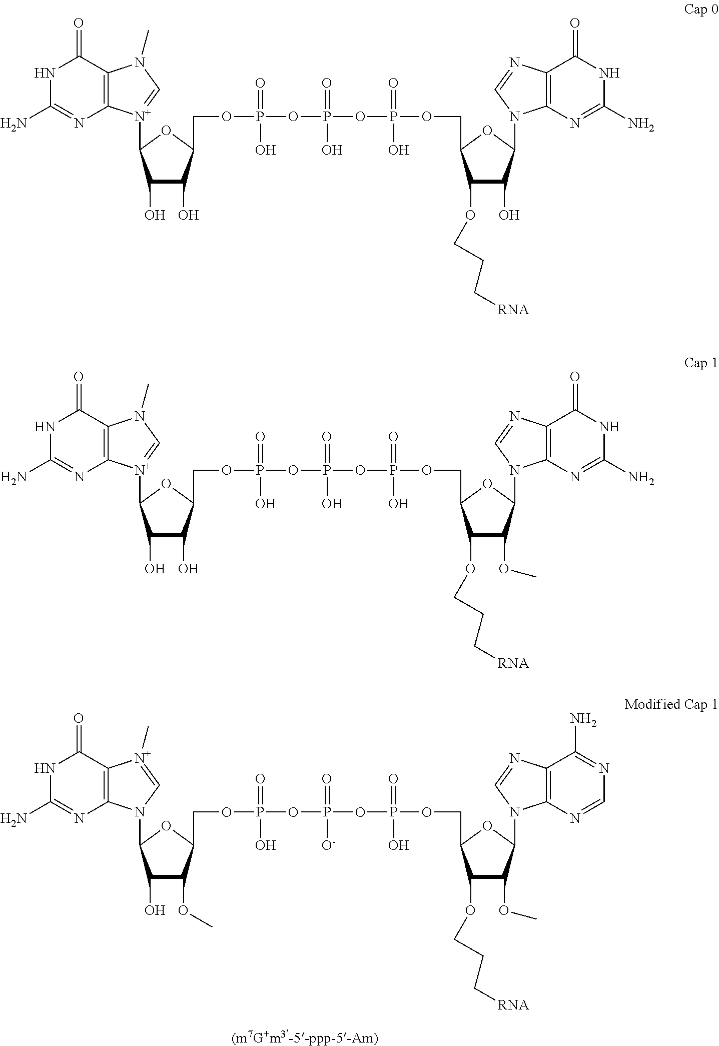

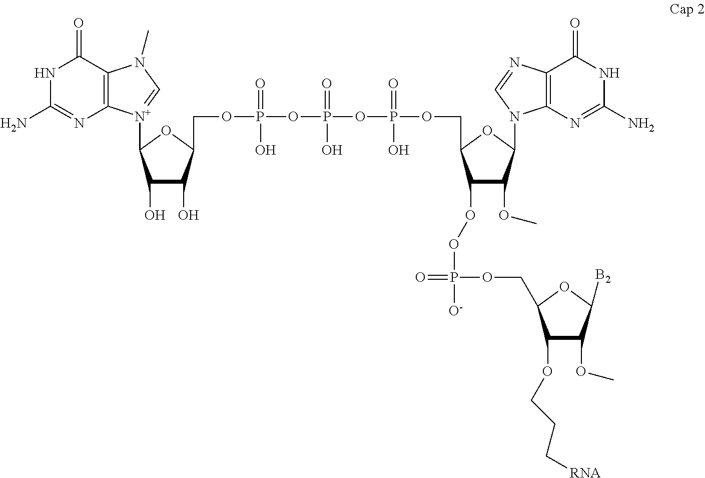

Without wishing to be bound by theory, it is thought that 5′ caps increase mRNA stability by protecting against degradation of the mRNA by 5′ exonucleases; that 5′ caps allow the ribosome to recognize the beginning of the mRNA; and that 5′ caps can improve translation efficiency by binding to the eukaryotic translation initiation factor 4E (eIF4E).

3′ poly (A) Tail

Without wishing to be bound by theory, it is also thought that modification of the mRNA by the addition of a 3′ poly (A) tail can improve translational activities and mRNA stability, including protecting mRNA from nuclease degradation. A poly (A) tail can, in preferred embodiments (for example when the open reading frame of the mRNA encodes a spike protein antigen of SARS-CoV-2) be added to the mRNA during in vitro transcription of the mRNA by including a poly (A) sequence in the DNA template. The tail size can be selected to optimise stabilisation and expression of the mRNA. Advantageously, in vitro transcription of mRNA from a DNA template can produce mRNA having a defined poly (A) tail length. In some embodiments, the poly (A) tail can have a length of between 100 and 200 nucleotides, for example between 120 and 150 nucleotides. In certain preferred embodiments, for example when the open reading frame of the mRNA encodes a spike protein antigen of SARS-CoV-2, a 110-nucleotide poly (A)-tail is provided having 30 adenosine residues separated by a 10-nucleotide linker sequence from a further 70 adenosine residues.

### Untranslated Regions (UTRs)

Untranslated regions are non-coding regions of the mRNA sequence, located at the upstream (5′ UTR) and downstream (3′ UTR) domains of the mRNA coding region. Without wishing to be bound by theory, it is thought that UTRs can assist with transcription regulation, as well as mRNA stability, and that UTRs impact translation efficiency through involvement in translation machinery recognition, recruitment, and mRNA trafficking. It is thought that UTRs can alter mRNA decay and translation efficiency through reactions with RNA binding proteins.

In certain preferred embodiments, for example when the open reading frame of the mRNA encodes a spike protein antigen of SARS-CoV-2, a 5′ UTR is provided which is derived from human alpha-globin RNA with an optimized Kozak sequence, and/or a 3′ UTR is provided comprising two sequence elements derived from the amino-terminal enhancer of split (AES) mRNA and the mitochondrial encoded 12S ribosomal RNA. Without wishing to be bound by theory, it is thought that such UTRs confer RNA stability and high total protein expression.

### Open Reading Frame (ORF)

As the term is used herein, the open reading frame (ORF) refers to the, or one of the, protein-encoding region of the nucleic acid (for example mRNA or pDNA). In some embodiments, the ORF sequence may include synonymous common codons (and/or codons having higher tRNA abundance) as replacements for rarer codons. It is thought that in this way, highly expressed genes can be translated using the same codons of the host, and/or guarantee the abundance of the relevant tRNA during the expression of the nucleic acid. However, having a higher translation rate of the nucleic acid may not always be preferred, as some proteins require a low translation rate for proper folding. In these circumstances, using codons with low frequency in ORF may yield higher quality protein products.

### Modified Nucleosides and Nucleotides

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides will also include modifications on the sugar moiety, e.g., wherein one or more of the hydroxyl groups are replaced with a halogen, an aliphatic group, or are functionalized as ethers, amines, or the like. Other modifications to nucleotides or polynucleotides involve rearranging, appending, substituting for, or otherwise altering functional groups on the purine or pyrimidine base which form hydrogen bonds to a respective complementary pyrimidine or purine, e.g., isoguanine, isocysteine, and the like. In some embodiments, the mRNA includes at least one, two, three or four modified nucleotides. For example, in some embodiments, the mRNA can include at least one N1-methyl-pseudouridine nucleoside, for example at least two, three, four, five, ten, fifteen or twenty N1-methyl-pseudouridine nucleosides.

In some embodiments, the nucleic acid (for example mRNA) includes one or more universal bases. As used herein, the term “universal base” refers to a nucleotide analogue that can hybridize to more than one nucleotide selected from A, U/T, C, and G. In some embodiments, the universal base can be selected from the group consisting of deoxyinosine, 3-ntiropyrrole, 4-nitroindole, 6-nitroindole, 5-nitroindole.

Without wishing to be bound by theory, it is thought that introducing modified nucleosides can help to modulate the innate immune response and/or increase nucleic acid stability. For example, in some embodiments, the uridine content of the mRNA can be replaced partially or entirely by N1-methyl-pseudouridine. In certain preferred embodiments, for example when the open reading frame of the mRNA encodes a spike protein antigen of SARS-CoV-2, the uridine content of the mRNA can be replaced entirely by N1-methyl-pseudouridine.

### Enzymes and Fragment or Fragments Thereof

According to all aspects of the invention relating to nucleic acid vector compositions, the composition may comprise one or more enzymes and/or one or more fragments thereof. The composition may comprise, in at least trace amounts, one or more of: linear DNA: one or more RNA polymerases and/or one or more fragments thereof; and one or more nucleoside triphosphates. Thus, in particular, the composition may comprise one or more RNA polymerases, and/or one or more fragments thereof. Optionally, the one or more RNA polymerases may be selected from one or more of: T7, SP6 and T3 RNA polymerase. If the nucleic acid is or comprises DNA, the composition may comprise, in at least trace amounts, linear DNA.

The composition may comprise, in at least trace amounts, enzymes and/or one or more fragments thereof, capable of degradation of the nucleic acid. However, the one or more particles comprising hydrolysable silicon may remove or sequester water molecules, thereby preventing the one or more enzymes, and/or one or more fragments thereof, from degrading the nucleic acid, on the basis that enzymes require an aqueous environment in order for them to catalyse the degradation of a nucleic acid.

### The One or More Particles Comprising Hydrolysable Silicon

According to all aspects of the invention, the one or more particles, which comprise hydrolysable silicon, may be pure silicon, or another hydrolysable silicon-containing material. If they are not pure silicon, they contain at least 50% by weight silicon, i.e. they comprise at least 50% by weight silicon atoms based on the total mass of atoms in the particles. For example, the silicon particles may contain at least 60, 70, 80, 90 or 95% silicon. The silicon particles preferably show a rate of hydrolysis, for example in PBS buffer at room temperature, of at least 10% of the rate of hydrolysis of pure silicon particles of the same dimensions. Assays for hydrolysis of silicon-containing material are widely known in the art (see, for example, WO2011/001456, incorporated by reference herein). Although particle of the invention may contain some silica, silica is not hydrolysable silicon and at least half of the silicon atoms in the particles are in the form of elemental silicon (or doped elemental silicon).

According to all aspects of the invention, the particles comprising hydrolysable silicon may be nanoparticles. Nanoparticles have a nominal diameter of between 5 and 400 nm, for example 50 to 350 nm, for example 80 to 310 nm, for example 100 to 250 nm, for example 120 to 240 nm, for example 150 to 220 nm, for example about 200 nm. They may be made of either pure silicon or a hydrolysable silicon-containing material. They are preferably porous. The nominal diameter referred to above, may refer to the mean diameter and at least 90% of total mass of particles in a sample of particles may fall within the size range specified. Particle size may be ascertained or confirmed by transmission electron microscopy (TEM), for example using the NIST-NCL Joint Assay Protocol, PCC-X, version 1.1, “Measuring the size of nanoparticles using TEM”, revised February 2010: https://tsapps.nist.gov/publication/get_pdf.cfm?pub_id=854083

In some preferred embodiments the nominal diameter may be below 100 nm, or below 80 nm, or below 70, 50 or 30 mn. In some embodiments the nominal diameter may be about 30 nm (for example between 20 and 40 nm) Particles comprising hydrolysable silicon can be made porous by standard techniques such as contacting the particles with a hydrofluoric acid (HF)/ethanol mixture and applying a current. By varying the HF concentration and the current density and time of exposure, the density of pores and their size can be controlled and can be monitored by scanning electron micrography and/or nitrogen adsorption desorption volumetric isothermic measurement.

If the particles are porous, their total surface area will be increased by virtue of their porosity. For example the surface area may be increased by at least 50% or at least 100% over the surface area of a corresponding non-porous particle. In many circumstances porous particles in accordance with all aspects of the invention will in reality have a much greater increase in total surface area by virtue of their porosity. According to certain embodiments the porosity is at least 30, 40, 50 or 60%. This means that, respectively, 30, 40, 50 or 60% of the particle volume in pore space. Preferred pore diameters range from 1 nm to 50 nm, for example from 5 nm to 25 nm.

### Optional Doping of Silicon

All aspects of the present invention optionally concern doped silicon containing material. The silicon may optionally be n-doped or p-doped. The invention includes embodiments in all aspects wherein the silicon is doped with one or more elements selected from Mg, P, Cu, Ga, Al, In, Bi, Ge, Li, Xe, N, Au, Pt. Most preferably the dopant is a p-dopant. Most preferably the dopant is boron. P-doped silicon is especially suitable for stabilising negatively charged nucleic acid. N-doped silicon may also be useful in indirectly stabilising negatively charged nucleic acids because they can protect lipids from degradation which may indirectly increase the stabilization and protection of the nucleic acid.

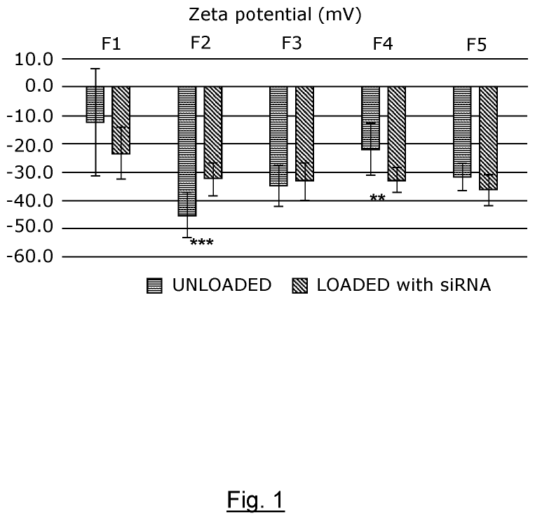

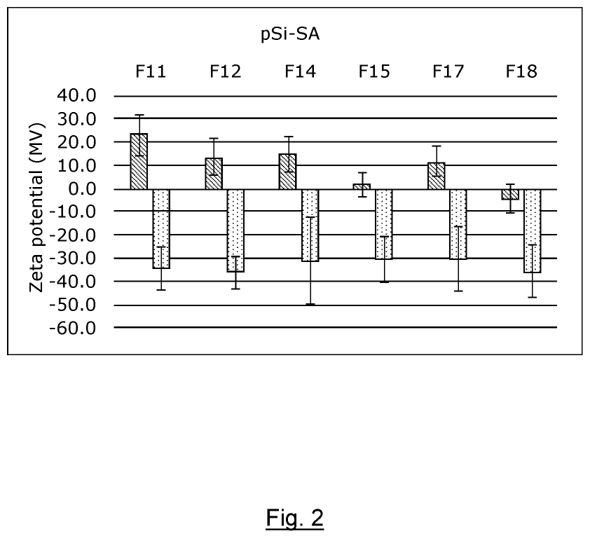

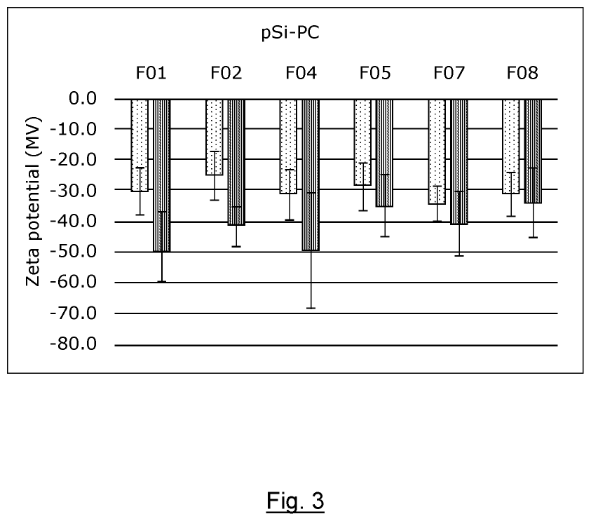

The manufacture of doped-silicon is well understood in the semiconductor industry and includes ion implantation and diffusion methods. Doped silicon is therefore readily available. Optionally, silicon can be doped by using a diffusion method to increase the amount of dopant present in the silicon. As an example of a diffusion method, silicon powder and a doping reagent (for example B203 for boron doping) are placed in a bowl, which is mixed and placed under an N₂ atmosphere and subjected to a temperature of between 1050° C. and 1175° C. for a few minutes to allow the dopant (for example boron) to diffuse into the silicon. FIGS. 1 and 2 show boron doped silicon produced by this method.

According to certain embodiments, doping of the silicon is heavy. Heavy boron doping is especially preferred. Heavy doping is understood to mean doping of at least 1×10¹⁵ dopant atoms per cm³. In some embodiments dopant is present at levels of at least 1×10¹⁶ dopant atoms per cm³, at least 1×10¹⁷ dopant atoms per cm³, at least 1×10¹⁸ dopant atoms per cm³, at least 1×10¹⁹ dopant atoms per cm³, or at least 1×10²⁰ dopant atoms per cm³.

When boron is used as the dopant, as is preferred, doping levels of 1×10¹⁵ dopant atoms per cm³, and 1×10²⁰ dopant atoms per cm³ correspond, respectively, to a resistivity of 20 mohm-cm, and 1 mohm-cm. The various aspects of the invention in which boron is the preferred dopant, do not exclude silicon which in addition to being doped, for example heavily doped, with boron, is also doped with other elements. According to preferred embodiments of all aspects of the invention, the majority dopant is boron.

### Ratio of Silicon to Nucleic Acid

Preferably, the ratio of silicon to nucleic acid (for example mRNA or pDNA) is between 0.01:1 and 1:8, for example between 1:1 and 1:6, 1:1 and 1:5, 1:1 and 1:4, or between 1:1 and 1:3. Preferably, the ratio of silicon to nucleic acid is between 1:1 and 1:3. Advantageously, this ratio of silicon to nucleic acid further stabilises the nucleic acid conveyed by the particle, thus optionally also affecting the rate of release of the nucleic acid.

Optional Nucleic Acid Association with Silicon

In some embodiments of all aspects of the invention, at least 70%, for example at least 80%, for example at least 90% of the nucleic acid (for example mRNA or pDNA) by weight present is associated with the one or more particles comprising hydrolysable silicon. By this, it is meant that nucleic acid is non-covalently associated with the one or more particles comprising hydrolysable silicon. Without wishing to be bound by theory, it is hypothesised that when this takes place, the random movement (also referred to as Brownian motion) of the nucleic acid may, in some embodiments, decrease: such that opportunities for the nucleic acid to be degraded are further reduced.

The optional association of the nucleic acid with the one or more particles comprising hydrolysable silicon may be correlated with, and/or governed by, the hydrolysis of the silicon. Thus, the rate at which the nucleic acid becomes bio-available may also be correlated with the hydrolysis of the silicon, thereby avoiding dose-dumping and/or ensuring gradual release of the nucleic acid over a suitably long period of time. Association between the nucleic acid and the one or more particles may, in some embodiments, further stabilise the nucleic acid. For example, it may result in charge stabilisation of the nucleic acid.

### Lipids

According to all aspects of the invention, the at least one lipid may comprise a cationic lipid: a helper lipid, e.g. a phospholipid: a structural lipid, e.g. a cholesterol-based lipid; and/or a polyethylene glycol (PEG) lipid.

The type of lipid or types of lipids used may affect the rate of degradation of the one or more silicon particles in vivo. For example, one or more lipid molecules may associate non-covalently with a surface or surfaces of the one or more silicon particles; this may be expressed as surface treating the one or more silicon particles with a lipid. For example, the presence of the at least one lipid has been found to allow for the rate of hydrolysis of the silicon to be controlled, such that the silicon hydrolyses to the bioavailable orthosilicic acid (OSA) degradation product rather than insoluble polymeric hydrolysis products. In particular, surface treating a silicon particle with a lipid has been found to have a beneficial effect on the surface charge of silicon particles, providing them with the requisite zeta potential to allow for improved stabilisation of the nucleic acid (e.g. mRNA), and optionally controlling the rate of release of the nucleic acid at a target site.

The lipids may include one or more of: phosphatidylcholine (PC), hydrogenated PC, stearylamine (SA), dioleoylphosphatidylethanolamine (DOPE), cholesteryl 3β-N-(dimethylaminoethyl) carbamate hydrochloride (DC)-cholesterol, 1,2-dioleoyl-3-trimethylammonium-propane (DOTAP) and derivatives of any thereof. In certain embodiments, the lipid comprises or consists of DOTAP. The type of lipid used to treat the surface of the nanoparticle may affect the stabilisation, optionally the rate of release, of the nucleic acid (for example mRNA or pDNA). In particular, where there is an association between lipid molecules and one or more silicon particles, there is a beneficial effect on the surface charge of the silicon particles, providing them with the requisite zeta potential to allow for improved stabilisation of nucleic acid, and optionally controlling the rate of nucleic acid (for example mRNA or pDNA) release at a target site. The presence of the at least one lipid may allow for the rate of hydrolysis of the silicon to be controlled, such that the silicon hydrolyses to the bioavailable orthosilicic acid (OSA) degradation product rather than insoluble polymeric hydrolysis products. Controlling the rate of hydrolysis of the silicon will affect how long the protection of the nucleic acid is sustained for.

According to all aspects of the invention, the lipid or lipids may have an average molecular weight in the range of from 500 to 1000 (for example, when the lipid contains one or more of a cationic lipid (for example, DTDTMA (ditetradecyl trimethyl ammonium), DOTMA (2,3-dioleyloxypropyl-1-trimentyl ammonium), DHDTMA (dihexadecyl trimethyl ammonium)) DOTAP, a helper lipid, a structural lipid and a PEG lipid, or is selected from one or more of PC, hydrogenated PC, SA, DOPE, DOTAP, DTDTMA, DHDTMA, DC-cholesterol, and derivatives thereof) the ratio of lipid (i.e. total lipid components) to silicon, before any extrusion or filtration process takes place, is between 1:1 and 20:1, for example between 1:1 and 18:1, 1:1 and 16:1, 1:1 and 11:1, 1:1 and 10:1, 1:1 and 9:1, 1:1 and 8:1, 1:1 and 13:1, 2:1 and 12:1, 2:1 and 11:1, 2:1 and 10:1, 2:1 and 9:1, 2:1 and 8:1, for example between 1:1 and 7:1, between 2:1 and 7:1, between 3:1 and 6:1, between 4:1 and 5:1. Without wishing to be bound by theory, this ratio of lipid to silicon may optionally provide a vesicle system able to control the release of, and stabilise nucleic acid (such as mRNA or pDNA) in contact with the particle of hydrolysable silicon and to facilitate the controlled release of the bioavailable degradation product of the silicon, OSA.

Advantageously, the lipid compound exerts a significant effect on the surface charge of the silicon particles. Particles comprising hydrolysable silicon treated with phosphatidylcholine (PC), phosphatidylethanolamine (PE) DOTAP and lecithin demonstrated a negative surface charge when zeta potential analysis was performed (ranging from −60 to −20 mV, with ratios of silicon:lipid ranging between 1:1 to 1:3). Particles surface treated with stearylamine demonstrated a positive zeta potential (ranging from 0 to 40 mV, with ratios of silicon:lipid ranging from 1:1 to 1:3). Using doped-silicon (for example p-doped silicon, for example boron-doped silicon as described elsewhere herein) may contribute to making the zeta potential more negative still. Controlling the surface charge of the silicon particles in this way may control their ability to remove or sequester water molecules and thus protect the nucleic acid from degradation.

The lipid or lipids component may, in some embodiments, be or comprise a phospholipid. The term “phospholipid” refers to a lipid comprising a fatty acid chain and a phosphate group. Phospholipids are typically neutral molecules in that they do not have an overall charge or may carry a negative charge, unlike a cationic lipid which is positively charged. Phospholipids are typically zwitterionic compounds comprising both positive and negatively charged components, but no overall charge. As such, phospholipids are typically classified as neutral lipids. Particularly suitable phospholipids are glycerophospholipids. Particularly suitable phospholipids are those in which the polar head group is linked to quaternary ammonium moieties, such as phosphatidylcholine (PC) or hydrogenated phosphatidylcholine. Another example of a phospholipid is DOPE (phosphatidyl ethanolamine or 1,2-dioleoyl-sn-glycero-3-phosphoethanolamine). The type of lipid may be selected depending on the nature of the formulation, with neutral or negatively charged phospholipids being preferred for aprotic formulations, while positively charged cationic lipids and small CH₃ chain lipids are preferred for protic formulations. The phospholipid may be or be derived from lecithin.

Preferably, the side chain(s) of the phospholipid is/are (an) aliphatic side chain(s) with 15 or more carbon atoms, or an ether side chain with 6 or more repeating ether units, such as a polyethylene glycol or polypropylene glycol chain. Lipids with ether side chains may be referred to as “PEG-lipids” or “PEG-ylated” lipids. The PEG-lipid may be a phospholipid such as 1,2-distearoyl-sn-glycero-3-phosphoethanolamine (DSPE) with a PEG side chain, e.g. DSPE-mPEG2000.

The lipid component may include one or more of phosphatidylcholine (PC), hydrogenated PC, stearylamine (SA), dioleoylphosphatidylethanolamine (DOPE), DOTAP, cholesteryl 3β-N-(dimethylaminoethyl) carbamate hydrochloride (DC)-cholesterol, and derivatives thereof. In certain embodiments the lipid component may consist substantially of phosphatidylcholine, hydrogenated phosphatidylcholine, stearylamine, or combinations thereof.

Doping of silicon changes the surface charge. Use of a p-dopant, in accordance with certain preferred embodiments, such as boron (which is preferred in many embodiments of the invention in all of its various aspects) will make the zeta potential more positive (i.e., less negative). A typical value of −40 mV for pure silicon will become about −25 mV when the silicon is doped with boron. It can therefore be understood that boron doped silicon can more easily achieve a positive zeta potential when treated with a cationic lipid. For example, treatment with stearylamine or DOTAP can achieve a value of about +20 mV to +60 mV. This means that a positive surface zeta potential may be achieved with a lower amount of cationic lipid or with a wider range of cationic lipids (including those which are less cationic than stearylamine and DOTAP). It also means that even if the cationic lipid degrades (“ages”) during storage resulting in a partial loss of positive charge of the lipid, the surface zeta potential of the particle will remain in a sufficiently positive range for longer.

It has been found that lipid to boron-doped silicon molar ratios of between 0.8:1 and 20:1 are particularly advantageous, for example 1:1, 6:1, 8:1, or 10:1, or 12:1 or 16:1.

The lipid or lipids component may, in some embodiments, be or comprise a phospholipid. The term “phospholipid” refers to a lipid comprising a fatty acid chain and a phosphate group. Phospholipids are typically neutral molecules in that they do not have an overall charge or they may carry a negative charge, unlike a cationic lipid which is positively charged. Phospholipids are typically zwitterionic compounds comprising both positive and negatively charged components, but no overall charge. As such, phospholipids are typically classified as neutral lipids. Particularly suitable phospholipids are glycerophospholipids. Particularly suitable phospholipids are those in which the polar head group is linked to quaternary ammonium moieties, such as phosphatidylcholine (PC) or hydrogenated phosphatidylcholine. Another example of a phospholipid is DOPE (phosphatidyl ethanolamine or 1,2-dioleoyl-sn-glycero-3-phosphoethanolamine). The type of lipid may be selected depending on the nature of the formulation, with neutral or negatively charged phospholipids being preferred for aprotic formulations, while positively charged cationic lipids and small CH₃ chain lipids are preferred for protic formulations. The phospholipid may be, or be derived from lecithin.

Preferably, the side chain(s) of the phospholipid is/are (an) aliphatic side chain(s) with 15 or more carbon atoms, or an ether side chain with 6 or more repeating ether units, such as a polyethylene glycol or polypropylene glycol chain. Lipids with ether side chains may be referred to as “PEG-lipids” or “PEG-ylated” lipids.

The lipid or lipids component may, in some embodiments, be or comprise a cationic lipid. The term “cationic lipid” refers to positively charged molecules having a cationic head group attached via a suitable spacer to a hydrophobic tail. Examples include DTDTMA (ditetradecyl trimethyl ammonium), DOTMA (2,3-dioleyloxypropyl-1-trimentyl ammonium), DOTAP, DHDTMA (dihexadecyl trimethyl ammonium) and stearylamine (SA). The positive charge is typically stabilised by a negative counter ion. In preferred embodiments, especially in relation to vaccine compositions, the cationic lipid is, or comprises DOTAP.

In certain embodiments, the lipid is selected from the group consisting of phosphatidylethanolamine (PE), phosphatidylcholine (PC), stearylamine (SA), or any combination thereof.

In certain embodiments the lipid may consist substantially of phosphatidylcholine, hydrogenated phosphatidylcholine, stearylamine, or combinations thereof.

In certain embodiments the lipid may consist of at least 5% by weight of hydrogenated phosphatidylcholine, for example at least 20 wt %, typically at least 30 wt % and especially at least 50 wt % hydrogenated phosphatidylcholine based on the total weight of the particle. It has been found that hydrogenated phosphatidylcholine to silicon molar ratios of between 0.8:1 to 5:1 are particularly advantageous, for example 1:1, 1.5:1, 2:1, 2.5:1, 3:1, 3.5:1, 4:1, or 4.5:1.

In certain embodiments the lipid may consist of at least 5% by weight phosphatidylcholine, for example at least 20 wt %, typically at least 30 wt % and especially at least 50 wt % phosphatidylcholine, based on the total weight of the particle. It has been found that phosphatidylcholine to silicon molar ratios of between 0.8:1 to 5:1 are particularly advantageous, for example 1:1, 1.5:1, 2:1, 2.5:1, 3:1, 3.5:1, 4:1, or 4.5:1.

In certain embodiments the lipid may consist of at least 5% by weight of stearylamine, for example at least 20 wt %, typically at least 30 wt % and especially at least 50 wt % stearylamine based on the total weight of the particle. It has been found that stearylamine to silicon molar ratios of between 0.8:1 to 5:1 are particularly advantageous, for example 1:1, 1.5:1, 2:1, 2.5:1, 3:1, 3.5:1, 4:1, or 4.5:1.

In certain embodiments the lipid may consist of PC and SA, preferably in a ratio by weight of PC:SA of from 1:1 to 20:1, more preferably 7:1 to 10:1, such as a ratio by weight of PC:SA of 72:8.

In certain embodiments the lipid may consist of DOPE, SA, and DC-cholesterol. The ratio by weight of DOPE:SA may be in a range of from 1:1 to 10:1, for example from 4:1 to 8:1. The ratio by weight of DOPE:DC-cholesterol may be in a range of from 1:1 to 5:1, for example from 1:1 to 3:1. The ratio by weight of SA:DC-cholesterol may be in a range of from 1:1 to 1:5, for example from 1:2 to 1:4. In some embodiments, the ratio by weight of DOPE:SA:DC-cholesterol may be 48:8:24.

In certain preferred embodiments the lipid may consist of DOTAP, DOPE and a PEG-lipid (such as mPEG2000-DSPE). The weight ratio of DOTAP:DOPE may be from 1:2 to 2:1, for example approximately 1:1. The ratio of DOTAP:PEG-lipid and DOPE:PEG-lipid may be 10:1 to 5:1, for example approximately 7:1. The total weight of ration of total lipid to silicon may be between 20:1 and 10:1, for example approximately 16:1.

The lipid component may be or comprise an ionizable lipid. The term “ionizable lipid” refers to lipids with a group capable of becoming positively charged typically having a Lewis base (hydrogen acceptor) head group attached, e.g. via some spacer, to a hydrophobic tail. Ionizable lipids typically include a head portion containing tertiary amine moieties. Ionizable lipids may be neutral at physiological pH but becomes cationic at lower pH, e.g. below pH 6.5, such as the pH found inside a vacuole as part of endosomal escape. Ionizable lipids are described, for example, in Nano Lett. 2020 Mar. 11; 20 (3): 1578-1589. Many vaccine platforms use lipid systems that are neutral at physiological pH and then become cationic only when endocytosed into an endosome and pH reduces to around pH 6.2, then the lipids that become cationic allow endosomal escape. Delivery systems comprising neutral lipids allows more of the particle to migrate from the muscle to the lymph node and become phagocytosed by dendritic cells. The ionisable lipid may be stabilised by association with the material of the solid biocompatible particles, e.g. by association with hydrolysable silicon.

In certain embodiments, the lipid is selected from the group consisting of phosphatidylethanolamine (PE), phosphatidylcholine (PC), stearylamine (SA), or any combination thereof.

In certain embodiments the lipid may consist substantially of phosphatidylcholine, hydrogenated phosphatidylcholine, stearylamine, or combinations thereof.

In certain embodiments the lipid may consist of at least 5% by weight of hydrogenated phosphatidylcholine, for example at least 20 wt %, typically at least 30 wt % and especially at least 50 wt % hydrogenated phosphatidylcholine based on the total weight of the particle. It has been found that hydrogenated phosphatidylcholine to silicon molar ratios of between 0.8:1 to 5:1 are particularly advantageous, for example 1:1, 1.5:1, 2:1, 2.5:1, 3:1, 3.5:1, 4:1, or 4.5:1.

In certain embodiments the lipid may consist of at least 5% by weight phosphatidylcholine, for example at least 20 wt %, typically at least 30 wt % and especially at least 50 wt % phosphatidylcholine, based on the total weight of the particle. It has been found that phosphatidylcholine to silicon molar ratios of between 0.8:1 to 5:1 are particularly advantageous, for example 1:1, 1.5:1, 2:1, 2.5:1, 3:1, 3.5:1, 4:1, or 4.5:1.

In certain embodiments the lipid may consist of at least 5% by weight of stearylamine, for example at least 20 wt %, typically at least 30 wt % and especially at least 50 wt % stearylamine based on the total weight of the particle. It has been found that stearylamine to silicon molar ratios of between 0.8:1 to 5:1 are particularly advantageous, for example 1:1, 1.5:1, 2:1, 2.5:1, 3:1, 3.5:1, 4:1, or 4.5:1.

In certain embodiments the lipid may consist of PC and SA, preferably in a ratio by weight of PC:SA of from 1:1 to 20:1, more preferably 7:1 to 10:1, such as a ratio by weight of PC:SA of 72:8.

In certain embodiments the lipid may consist of DOPE, SA, and DC-cholesterol. The ratio by weight of DOPE:SA may be in a range of from 1:1 to 10:1, for example from 4:1 to 8:1. The ratio by weight of DOPE:DC-cholesterol may be in a range of from 1:1 to 5:1, for example from 1:1 to 3:1. The ratio by weight of SA:DC-cholesterol may be in a range of from 1:1 to 1:5, for example from 1:2 to 1:4. In some embodiments, the ratio by weight of DOPE:SA:DC-cholesterol may be 48:8:24.

### Polycationic Nucleic Acid-Binding Component

The compositions of the invention optionally further comprise a polycationic nucleic acid-binding component. The term “polycationic nucleic acid-binding component” is well known in the art and refers to polymers having at least 3 repeat cationic amino acid residues or other cationic unit bearing positively charged groups, such polymers being capable of complexion with a nucleic acid under physiological conditions. An example of a nucleic acid-binding polycationic molecule is an oligopeptide comprising one or more cationic amino acids. Such an oligopeptide may, for example, be an oligo-lysine molecule, an oligo-histidine molecule, an oligo-arginine molecule, an oligo-ornithine molecule, an oligo diaminopropionic acid molecule, or an oligo-diaminobutyric acid molecule, or a combined oligomer comprising or consisting of any combination of histidine, arginine, lysine, ornithine diaminopropionic acid, and diaminobutyric acid residues. Further examples of polycationic components include dendrimers and polyethylenimine.

### Amino Acids

All aspects of the invention may include the additional optional presence of one or more amino acids. In its broadest sense, the term “amino acid” encompasses any artificial or naturally occurring organic compound containing an amine (—NH₂) and carboxyl (—COOH) functional group. It includes an a, B, Y and 8 amino acid. It includes an amino acid in any chiral configuration. According to some embodiments (for example, when the silicon-containing particles of the invention are formulated with one or more of PC, hydrogenated PC, SA, DOPE, DOTAP, DC-cholesterol, and derivatives thereof) the amino acid is preferably a naturally occurring a amino acid. It may be a proteinogenic amino acid or a non-proteinogenic amino acid (such as carnitine, levothyroxine, hydroxyproline, ornithine or citrulline). In preferred embodiments the amino acid comprises arginine, histidine, or glycine, or a mixture of arginine and glycine. In particularly preferred embodiments, the amino acid comprises glycine. Such amino acids may function to stabilise the silicon particle and control the hydrolysis of silicon both in storage and in vivo.

Treating the lipid-treated silicon particles with an amino acid can also provide a beneficial stabilising effect on the nucleic acid. Treating lipid-treated silicon particles with amino acids has been shown to stabilise nucleic acids in biological fluids, for example in ocular tissues and plasma and tissue fluid. Lipid-treated particles formulated with an amino acid in this manner may be particularly suitable for delivery to the body, for example delivery by transcutaneous injection.

### Peptide

In addition to the amino acids described above, in all aspects of the invention there may be included a peptide containing a cell surface receptor-, for example integrin-, recognition sequence that confers a degree of cell specificity to the particle. The peptide may have a “head group” containing a cell surface receptor recognition sequence and additionally a “tail” that can bind non-covalently to the nucleic acid (for example mRNA or pDNA) and/or bind to the silicon.

### Ratio of Amino Acid(s) to Silicon

Preferably, when amino acid(s) is/are present, the ratio of amino acid to silicon is between 0.05:1 and 2:1, for example between 0.05:1 and 1.8:1, 0.05:1 and 1.6:1, 0.05:1 and 1.4:1, 0.05:1 and 1.2:1, 0.05:1 and 1:1, 0.05:1 and 0.9:1, 0.05:1 and 0.8:1, 0.05:1 and 0.6:1, 0.05:1 and 0.5:1, 0.05:1 and 0.4:1, 0.05:1 and 0.3:1, 0.05:1 and 0.2:1, preferably between 0.2:1 and 0.8:1, especially between 0.3:1 and 0.7:1. The ratio of amino acid to silicon may be between 0.05:1 to 0.4:1, for example between 0.08:1 and 0.35:1, especially 0.09:1 to 0.32:1. Advantageously, tuning the ratio of amino acid to silicon further stabilises the nucleic acid (for example mRNA or pDNA).

In some embodiments, the amino acid is a combination of arginine and glycine, wherein the ratio of Arg:Gly is between 1:0.6 and 3:1, for example between 1:0.8 and 2.5:1, for example between 1:1 and 2:1.

According to other embodiments of all aspects of the invention the particles are formulated with arginine. Preferably, the ratio of arginine to silicon is between 0.05:1 to 0.4:1, for example between 0.08:1 and 0.35:1, especially 0.09:1 to 0.32:1.

According to other embodiments of all aspects of the invention the particles are formulated with glycine. Preferably, the ratio of glycine to silicon is between 0.05:1 to 0.5:1, for example between 0.08:1 and 0.45:1, especially 0.09:1 to 0.42:1.

Preferred amino acids for use with all aspects of the invention include arginine, glycine, proline, lysine and histidine and mixtures of two or more thereof.

### Non-Reducing Disaccharide

According to all aspects of the invention there is optionally included compositions and related produces and methods at least one non-reducing disaccharide. The non-reducing disaccharide may optionally be selected from sucrose, trehalose, raffinose, stachyose and verbascose or mixtures of any thereof, most preferably the non-reducing disaccharide is trehalose, or a mixture comprising trehalose.

The non-reducing disaccharide (for example, trehalose or a mixture comprising trehalose) is optionally present at a weight ratio to silicon of at least 1:1000, at least 1:100, at least 1:50, at least 1:10, at least 1:1, or at least 1:0.5. Preferably, the non-reducing disaccharide is trehalose which is optionally present a weight ratio to silicon of at least 1:1000, at least 1:100, at least 1:50, at least 1:10, at least 1:1, or at least 1:0.5.

It is postulated that non-reducing disaccharides, especially trehalose, may act as a desiccation-protectant. Non-reducing disaccharides may act as a cocoon that traps nucleic acid molecules inside a glassy sugar matrix such that the movement is restricted by the sugar matrix. Trehalose may be particularly effective due to its ability to transit between one crystalline form and another, without relaxing its structural integrity and/or due to its particularly high glass transition temperature compared to other disaccharides such as sucrose. In addition in amorphous trehalose, local pockets of crystalline dihydrate exist, which trap residual water molecules, immobilizing them, which is of value to prevent nucleic acid degradation especially when water is relatively scarce. Thus, the water-sequestering effect of the silicon particles may be enhanced. An additional advantage of including a non-reducing disaccharide, such as trehalose, is that the presence of the non-reducing disaccharide facilitates resuspension of a pharmaceutical composition which is a powdered material.

### Especially Preferred Combinations

According to all aspects of the invention, especially preferred embodiments relate to doping which is boron doping (especially heavy boron doping as defined above) wherein the nucleic acid is an RNA, especially an mRNA encoding an antigen of an mRNA vaccine, and the lipid is or comprises an ionisable lipid, for example one or more cationic lipids, such as DOTAP, and/or one or more zwitterionic lipids, such as one or more phospholipids, such as DOPE: or both (for example, wherein the lipid comprises DOTAP or wherein the lipid comprises both an ionisable lipid and DOTAP).

### Further Components and Features

According to preferred embodiments of all aspects of the invention there may additionally be present one or more further components, including transfection reagents.

In its broadest sense, “transfection reagents” are agents that facilitate the introduction of naked or purified nucleic acids into eukaryotic cells. For example, some transfection reagents are agents that facilitate the induction of mRNA into eukaryotic cells.

According to other embodiments of all aspects of the invention, the transfection reagents may be lipofection (liposome transfection) reagents, dendrimers, a HEPES-buffered saline solution (HeBS) containing phosphate ions combined with a calcium chloride solution, or cationic polymers such as diethylaminoethyl-dextran (DEAE dextran) or polyethylenimine (PEI).

In preferred embodiments, the transfection reagent is a lipofection reagent, such as lipofectamine.

### Pharmaceutical Compositions

According to the third aspect of the invention, there is provided a pharmaceutical composition comprising the nucleic acid vector composition of the first aspect of the invention, wherein the pharmaceutical composition is a vaccine composition.

In some embodiments, nucleic acid (for example in vitro transcribed mRNA or pDNA) has a half-life in the pharmaceutical composition at 4° C. of at least 3 months, for example at least 4, 5 or 6 months.

In some embodiments, pharmaceutical and vector compositions of the invention are in a form suitable for intramuscular injection. Pharmaceutical and vector compositions of the invention may comprise excipients, including but not limited to: preservatives, cryoprotectants and immune adjuvants. As used herein, the term adjuvant may refer to a substance that modulates, for example increases, the immune response of a subject to the vaccine composition. By way of non-limiting example, an adjuvant may be or may comprise one or more lipids, proteins, CpG oligodeoxynucleotides, and/or other molecular adjuvants. The pharmaceutical and vector compositions may in some embodiments comprise one or more buffer components. The pharmaceutical and vector compositions may in some embodiments comprise trometamol. The pharmaceutical and vector compositions may in some embodiments comprise trometamol hydrochloride. The pharmaceutical and vector compositions may in some embodiments comprise acetic acid. The pharmaceutical and vector compositions may in some embodiments comprise sodium acetate trihydrate. The pharmaceutical and vector compositions may in some embodiments comprise potassium chloride. The pharmaceutical and vector compositions may in some embodiments comprise potassium dihydrogen phosphate. The pharmaceutical and vector compositions may in some embodiments comprise sodium chloride. The pharmaceutical and vector compositions may in some embodiments comprise disodium hydrogen phosphate dehydrate. The pharmaceutical and vector compositions may in some embodiments comprise sucrose. It will be appreciated that the pharmaceutical vector compositions of the invention may optionally be diluted with saline before administration by intramuscular injection. In some embodiments the pharmaceutical vector compositions may be a powder. In some embodiments the pharmaceutical and vector compositions may comprise a liquid.

In the pharmaceutical and vector composition of the invention, the nucleic acid (for example the in vitro transcribed mRNA or pDNA) can be protected from enzymatic degradation by the one or more particles comprising hydrolysable silicon, which sequester or remove water as described herein.

### Preparation of Particles Comprising Hydrolysable Silicon

The particles for use in the invention may conveniently be prepared by techniques conventional in the art, for example by milling processes or by other known techniques for particle size reduction. The silicon-containing particles may be made from sodium silicate particles, colloidal silica or silicon wafer materials. Macro, micro or nano scale particles are ground in a ball mill, a planetary ball mill, or other size reducing mechanism. The resulting particles may be air classified or sieved to recover particles of a uniform required size. It is also possible to use plasma methods and laser ablation for the production of particles. Porous particles may be prepared by methods conventional in the art, including the methods described herein.

### Preparation of Particles Comprising Doped Hydrolysable Silicon

An example specification of a boron-doped silicon for use according to the invention is: single side polished Wafer, CZDiameter: 150±0.2 mmOrientation: (100)±1° Type: p/boronresistivity: 0.014±25% Ohm cm. Close to 5×1018 atoms/^{cm3}Primary flat: 57,50±2.5 mmPrimary flat1 Location: D<100> to {110}Thickness: 675±15 μmpacking: Ultrapak Shipping CassetteTTV:<=18 μmTIR: <=5 μmSuch boron doped silicon is available commercially, for example from Nanografi, Jena, Germany or Si-Mat, Germany.

### Preparation of Pharmaceutical and Vector Compositions

Pharmaceutical compositions and vector compositions of the invention may be produced by bringing together the components. In some embodiments, this bringing together may merely involve mixing solutions of the components, for example under conditions leading to their complexation. The bringing together may involve contacting the nucleic acid with the particles of silicon containing material prior to the addition of the lipid components. Addition of the lipid components after contact of the nucleic acid with the silicon containing particles may facilitate the formation of a lipid shell as described herein, encapsulating a core, the core comprising both the one or more particles and the nucleic acid.

It has been found to be advantageous, and is therefore preferred in some embodiments that the silicon containing particles of the invention are activated before being bought into contact with other components of the invention. Activation may be carried out by dispersing the particles (e.g. porous doped-silicon nanoparticles) in a volatile alcohol or other volatile solvent (such as chloroform, methanol, ethanol or propanol, e.g. methanol) prior to bringing them into contact with other components of the invention.

Typically, silicon particles are mixed with the non-reducing disaccharide, if present, the nucleic acid and optional amino acids and then contacted with the lipid components to form lipid-silicon particles in which the nucleic acid is associated with the particle within a lipid compartment.

It is postulated that a delivery system comprising silicon containing particles, especially particles comprising porous hydrolysable doped-silicon, increase the stability of a nucleic acid, by sequestering water molecules that otherwise would react with the nucleic acid, in a hydrolysis reaction catalysed by an enzyme present in the composition.

### Lyophilisation

In certain embodiments, the pharmaceutically or vector composition is optionally lyophilised, optionally lyophilised with a cryoprotectant and/or a lyoprotectant, such as one or more sugars, such as sucrose and/or trehalose; for example to form a powder. Lyophilisation removes water, and enables a dry powder to be produced. Such a dry powder may optionally be dispersed for example in a hydrogel. Other physical forms of the compositions of the invention include liquid forms and frozen forms. In some embodiments the composition is provided in a delivery device, for example in an injection device such as a syringe or a multiplicity of microneedles.

### EXAMPLES

Various aspects and embodiments of the invention are now described with reference to the following non-limiting examples.

### Example 1: Preparation of Silicon Nanoparticles (SiNP)

Single side polished silicon wafers were purchased from Si-Mat, Germany. All cleaning and etching reagents were clean room grade. Etched silicon wafers were prepared by anodically etching Si in a 1:1 (v/v) mixture of pure ethanol and 10% aqueous HF acid for 2-10 min at an anodic current density of 80 mA/cm². After etching, the samples were rinsed with pure ethanol and dried under a stream of dry high-purity nitrogen prior to use.

Etched silicon wafers, P+ type or N− type, were crushed using a milling ball and/or a pestle & mortar.

### Specifications of Silicon Wafer

single side polished Wafer, CZ
Diameter: 150±0.2 mm
Orientation: (100)±1°
Type: p/boron
resistivity: 0.014±25% Ohmcm. Close to 5×10\hat{ }18 Atoms/cm\hat{ }3.
Primary flat: 57,50±2.5 mm
Primary flat1 Location: D<100> to {110}
Thickness: 675±15 μm
packing: Ultrapak Shipping Cassette
TTV: <=18 μm
TIR: <=5 μm

The wafers are around 40% porosity and up to 50 μm thick.

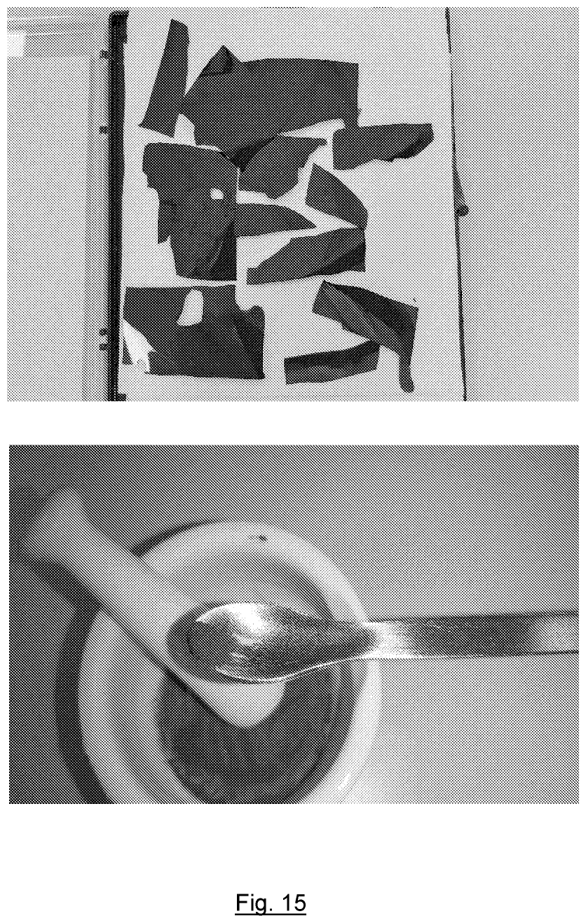

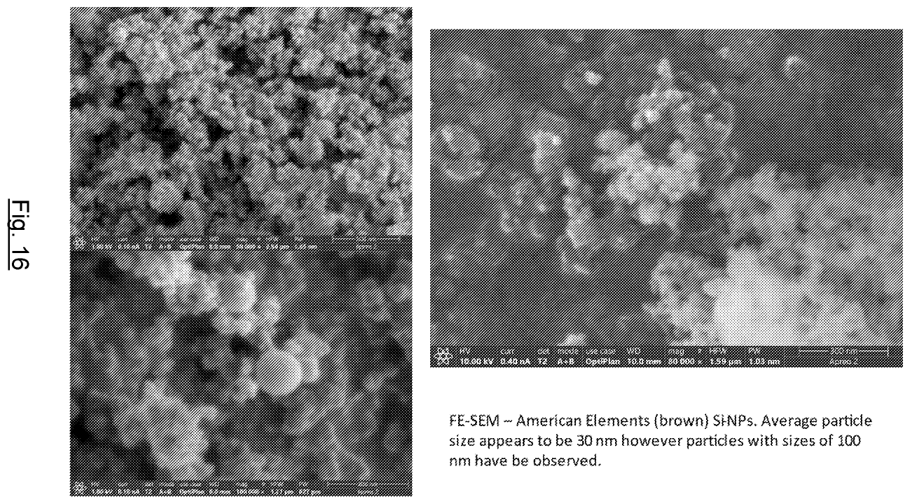

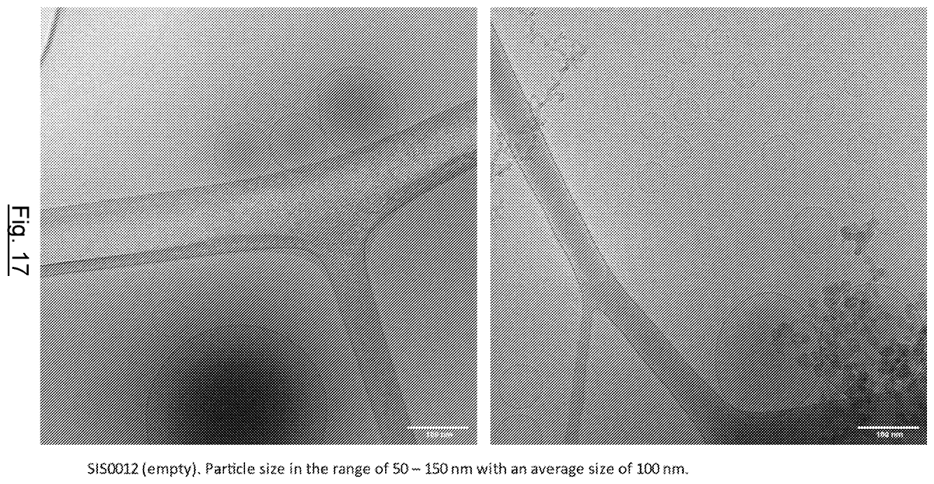

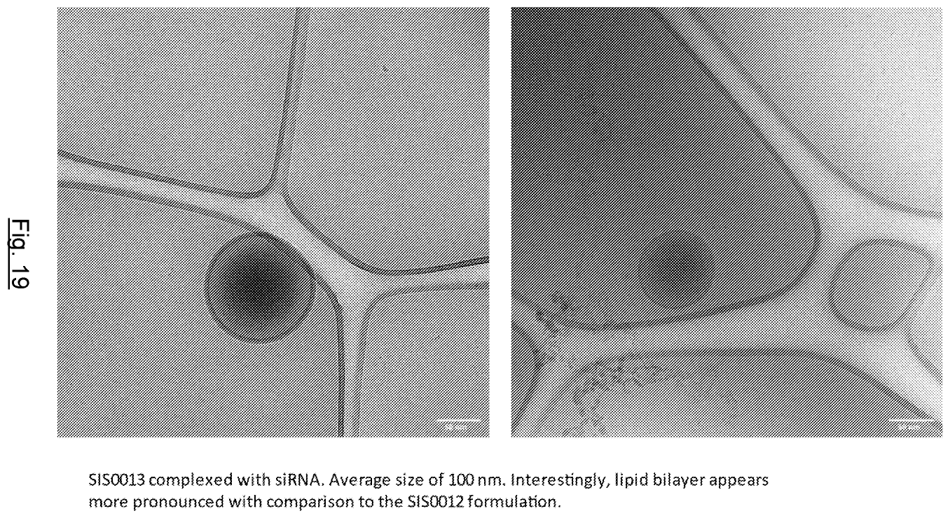

They were manually milled using a ball mill or pestle and mortar. The appearance before and after milling is shown in FIG. 15. FIG. 16 shows an SEM image of the silicon particles which was used to assess their size. FIGS. 17, 18 and 19 are TEM images of the particles (FIG. 17, silicon alone;

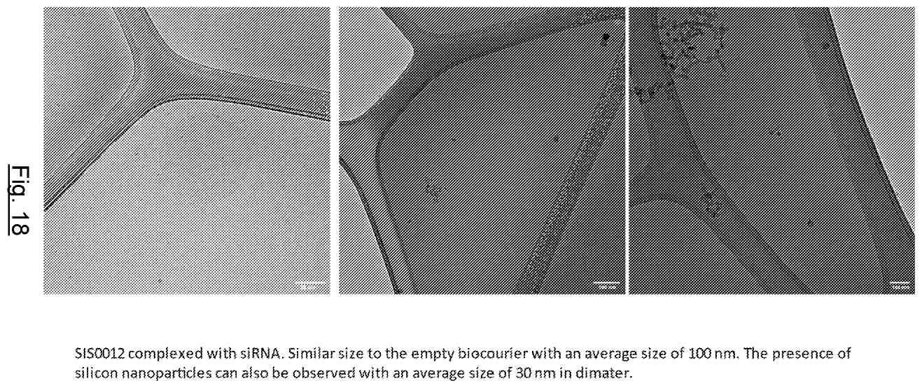

FIG. 18, silicon particle plus lipid and other components as “Biocourier” (see below for definition) and siRNA (SIS0012); FIG. 18, as FIG. 17 but produced with boron-doped silicon as SIS0013).

The fine powder was sieved using a Retsch™ sieve shaker AS 200 with gauge 38 μm. Uniform and selected particle size selection (20-100 μm) was achieved by the aperture size of the sieve. The particle sizes were measured by the quantachrome system and PCS from Malvern instruments. Samples were kept in a closed container until further use.

Nano silicon powder was also obtained from Sigma Aldrich and Hefei Kaier, China. The particle size was measured by PCS and the size of the particles was recorded (size was range between 20-100 nm) before being subjected to loading and etching.

500 mg of 100 nm diameter porous silicon nanoparticles were mixed with 250 ml ethanol and stirred using a magnet bar for 30 minutes. The solution was then centrifuged for 30 minutes at 3000 rpm. The supernatant was discarded and the nanoparticles were washed in 5 ml of distilled water and transferred to a round bottomed flask. The contents of the flask were frozen (2 hours at −25° C.). The frozen nanoparticles were freeze-dried using a freeze dryer overnight. The resultant dry powder were activated silicon nanoparticles.

### Example 2: Preparation of Lipid-Functionalised siRNA-SiNP Formulations

### Materials

Activated silicon nanoparticles (SiNP) prepared according to Example 1.

Nuclease-free water.

Chloroform.

Lipids: Stearylamine (SA), cat. 305391 (Sigma-Aldrich):
L-α-phospatidylcholine from egg yolk (PC), cat. 61755 (Sigma-Aldrich);
Dioleoyl L-α-phosphatidylethanolamine (DOPE), cat. P1223 (Sigma-Aldrich);
N-(2-dimethylaminoethyl) carbamate cholesterol (DC-Chol), cat. 92243 (Sigma-Aldrich). siRNA: nonspecific NSC4 (customised siRNA duplex, Eurogentec);
targeted siLUC (customised siRNA oligonucleotide duplex, Eurogentec); FAM-tagged siRNA (green siGlo, Dharmacon).

### Equipment

Rotary evaporation system, vortex, bath sonicator, water bath, round bottom flasks, universal test tubes, Eppendorf tubes, micropipettes, freeze drier system, ZetaSizer, high speed centrifuge, NanoDrop spectrophotometer, fluorescence reader.

### Procedure

N.B. For fluorescent siRNA (siGlo) light exposure was avoided during the procedure (e.g. cover flasks and tubes were covered with aluminium foil).

Preparation of Component 1: siRNA-SiNP Mix
1. Prepare a filtered solution of SiNP (0.2 mg/ml) in nuclease-free water.
2. Aliquot 700 μl of the above solution into 8 Eppendorf microtubes so that each contains 140 μg of SiNP.
3. To each aliquot add siRNA and glycine and adjust volume to 1.4 ml with nuclease-free water:

TABLE 1

| | | siRNA | | | | nuclease- | |
|---|---|---|---|---|---|---|---|
| | | non-specific | targeted | fluorescent | | free water | |
| | SiNPs | 100 μM | 100 μM | 100 μM | glycine | (up to | |
| aliquot ID | 0.2 mg/ml | NSC4 | siLUC | siGlo | 1 mg/ml | 1.4 ml) | |
| 1^{st} | 700 μl | 52.5 μl | — | — | 70 μl | 577.5 | μl |
| 2^{nd} | 700 μl | — | 52.5 μl | — | 70 μl | 577.5 | μl |
| 3^{rd} | 700 μl | — | — | 52.5 μl | 70 μl | 577.5 | μl |
| 4^{th} | 700 μl | — | — | — | 70 μl | 630 | μl |

4. Mix the tubes thoroughly and incubate samples 1 hr at room temperature with agitation.

### Preparation of Component 2: Lipid Film

1. Dissolve each lipid component (SA, PC, DOPE, DC-Chol) in chloroform to a concentration of 0.2 mg/ml.
2. Transfer the desired amount of each lipid into a small round bottom flask and mix thoroughly. Prepare each lipid mixture in 8 replicates (in 8 flasks):
   a. Lipid base DS61
      68 μg of DOPE (340 μl)
      12 μg of SA (60 μl)
   b. Lipid base PS91
      72 μg of PC (360 μl)
      8 μg of SA (40 μl)
   c. Lipid base DSC613
      48 μg of DOPE (240 μl)
      8 μg of SA (40 μl)
      24 μg of DC-Chol (120 μl)
   d. Lipid base PDS1051
      50 μg of PC (250 μl)
      25 μg of DOPE (125 μl)
      5 μg of SA (25 μl)
   e. Lipid base PDS1052
      48 μg of PC (240 μl)
      23 μg of DOPE (115 μl)
      9 μg of SA (45 μl)
   f. Lipid base PDSC10514
      40 μg of PC (200 μl)
      20 μg of DOPE (100 μl)
      4 μg of SA (20 μl)
      16 μg of DC-Chol (80 μl)
3. Carefully evaporate the solvent using a rotary evaporation system to form a thin lipid film. Place the dried lipids under vacuum to remove residual solvent.
   Functionalisation of siRNA-SiNP (Component 1) with Lipids (Component 2)
1. Dissolve thin lipid film (component 2) with siRNA-loaded or empty Si-nanoparticles samples (component 1). To each flask containing lipid film (a, b, c, d, e, f) add 200 μl of either 1^{st}, 2^{nd}, 3^{rd} or 4^{th} silicon nanoparticle/siRNA mix:

TABLE 2

| SIS005- | SIS005- | SIS005- | SIS005- | SIS005- | SIS005- |
|---|---|---|---|---|---|
| DS61G | PS91G | DSC613G | PDS1051G | PDS1052G | PDSC10514G |
| a-1^{st} | b-1^{st} | c-1^{st} | d-1^{st} | e-1^{st} | f-1^{st} |
| a-2^{nd} | b-2^{nd} | c-2^{nd} | d-2^{nd} | e-2^{nd} | f-2^{nd} |
| a-3^{rd} | b-3^{rd} | c-3^{rd} | d-3^{rd} | e-3^{rd} | f-3^{rd} |
| a-4^{th} | b-4^{th} | c-4^{th} | d-4^{th} | e-4^{th} | f-4^{th} |

2. Adjust volume of solutions to 400 μl by adding 200 μl of nuclease-free water to each flask.
3. Cover each flask with Parafilm, mix the content thoroughly and incubate at RT for 1 hr.
4. Vortex to completely dissolve the lipid film. Subject flasks to bath sonication for 15 sec to help dissolve the lipids.
5. Transfer all the content of each flask to Eppendorf microtubes in separate.
6. Place all tubes in the freezer (−20° C.) and keep them for at least 3 hours (or overnight).
7. Remove samples from the freezer and put them into 30° C. water bath for 10 min. Cool down at RT and vortex thoroughly.
8. Repeat freeze-thaw (step 5 and 6) two more times.
9. Keep all samples in the freezer (−20° C.) until assayed.
10. Each loaded sample contains [10 μg siRNA: 20 μg SiNP (silicon nanoparticles): 80 μg lipid base: 10 μg glycine] in 400 μl volume.

The components of the resulting formulations are shown in Table 3 below:

TABLE 3

| | siRNA | | | |
|---|---|---|---|---|
| Formulation | siLUC/NSC4/siGlo | siNPs | Lipids | Glycine |
| SIS005- | 10 μl | 20 μl | 80 μl | 10 μl |
| DS61G | | | [68 μl DOPE + 12 μl SA] | |
| SIS005- | 10 μl | 20 μl | 80 μl | 10 μl |
| PS91G | | | [72 μl PC + 8 μl SA] | |
| SIS005- | 10 μl | 20 μl | 80 μl | 10 μl |
| PDS1051G | | | [50 μl PC + 25 μl DOPE + | |
| SIS005- | 10 μl | 20 μl | 5 μl SA] 80 μl | 10 μl |
| PDS1052G | | | [48 μl PC + 23 μl DOPE + | |
| SIS005- | 10 μl | 20 μl | 9 μl SA] 80 μl | 10 μl |
| PDSC10514G | | | [40 μl PC + 20 μl DOPE + | |
| | | | 4 μl SA + 16 DC-Chol] | |
| SIS005- | 10 μl | 20 μl | 80 μl | 10 μl |
| DSC613G | | | [48 μl DOPE + 8 μl SA + | |
| | | | 24 μl DC-Chol] | |

Preparation of Amino Acid, Lipid, Lipofectamine-Loaded siRNA Nanoparticles

Formulations SIS005-PS91 and SIS005-DS61 (with glycine) were each prepared by dissolving thin films of liposomes-forming material in an aqueous suspension of silicon nanoparticles containing siRNA. The mixture was then triple freeze-thawed, and tested for transfection efficiency on HCES cells in vitro. In parallel, the SIS005-PDS1051 formula were tested after further modifications aimed to introduce more positive charge (as it was shown to have a negative zeta-potential) which is believed to be preferable for the siRNA delivery system for the cornea. For this purpose, the ratio of cationic lipid in the formulation was increased.

The colloidal stability was evaluated by the dynamic light scattering method in parallel to zeta-potential measurements, for both unloaded and loaded formulations. In addition, the encapsulation efficiency of siRNA was determined by a spectrophotometric method. The efficiency of transfection (internalization into the cells) will be assessed in human corneal epithelial cells by flow cytometry followed by the dual luciferase assay which was performed to determine knockdown in vitro after treatment with siRNA-loaded formulations.

### Characterization of Silicon-Nanoparticle/Lipid Formulations

### Encapsulation Efficiency

The siRNA encapsulation efficiency was investigated by determination of encapsulation efficiency after separation of loaded particles from free (unbound) siRNA by high speed centrifugation.
1. Collect 50 μl of each sample in a centrifuge microtube.
2. Centrifuge all samples at 21,000 g for 30 min.
3. Transfer 25 μl (upper half part) of supernatant from each sample to separate tubes (denote as S1) and store at 4° C. until assayed (avoid light exposure in case of siGlo-loaded samples).
4. Add 25 μl of 2% SDS to pellet samples (with remaining supernatant) to disrupt lipid bilayer and release bound siRNA.
5. Centrifuge the tubes again at 21,000 g for 30 min and collect supernatant (denoted as S2).
6. Measure absorbance (OD) at 260 nm of all supernatant S1 and S2 samples using NanoDrop spectrophotometer. For siGlo-loaded samples, measure fluorescence intensity (FI) in both S1 and S2 supernatant samples.
7. Calculate encapsulation efficiency (EE %):

$$EE \% = ( 1 - ( \frac{2 \times {OD}_{S 1}}{{OD}_{S 1} + 2 \times {OD}_{S 2}} ) ) \times 100 \%$$

where OD_{S1}-Absorbance (260 nm) of supernatant S1 (after 1st centrifugation) OD_{S2}-Absorbance (260 nm) of supernatant S2 (after 2nd centrifugation) For siGlo-loaded samples use FI_{S1} and FI_{S2} instead of OD_{S1} and OD_{S2}, respectively.
8. The concentration of siRNA in supernatant samples and the amount of siRNA entrapped by silicon nanoparticle formulation can be determined using siRNA calibration curve (as described previously).

### ZetaSizer Measurements

The colloidal stability was evaluated by a dynamic light scattering method in parallel to zeta-potential measurement. Measurements were carried out on unloaded (empty) formulation samples as well as loaded with siLUC/NSC4.
1. Collect 200 μl of each sample and dilute with nuclease-free ultrapure water to the total volume of 1 ml.
2. Load samples to folded capillary cells.
3. Read samples using ZetaSizer: particle dimensions, polydispersity index and zeta potential. Run each measurement in triplicate. Data is shown in FIGS. 1, 2, 3 and 4.

### Transfection Efficiency in HCES Cells

The efficiency of transfection (internalization into the cells) was assessed in human corneal epithelial cells by flow cytometry. For the purpose of this study, we use formulation samples loaded with fluorescently tagged siRNA probe (siGlo).
1. Seed 2×10⁵ HCES cells per well (on 12-well plate) in 1 ml of DMEM enriched with 10% FBS and grow for 24 hours (until 80% confluence) at standard conditions.
2. Change medium for 950 μl per well of fresh DMEM enriched with 10% FBS.
3. Adjust siGlo-loaded silicon nanoparticle/lipid samples to room temperature.
4. Prepare control sample with Lipofectamine transfection reagent by mixing 6 μl of Lipofectamine RNAiMAX reagent with 3 μl of siGlo stock solution (100 μM) in 151 μl of OptiMEM and incubate 15 min at room temperature.
5. Treat cells in 3 replicates by adding 53.3 μl of each siGlo-loaded formulation or LPF-control per well to obtain 0.1 μM siGlo concentration.
6. Grow cells for 24 hours at standard conditions (37° C. with 5% CO₂).
7. Discard medium and wash wells with 500 μl of PBS.
8. Add 300 μl of Trypsin-EDTA and incubate plates at 37° C. for 10 minutes.
9. Immediately add 300 μl of DMEM enriched with 10% FBS to stop trypsination.
10. Transfer the cells to separate microtubes and centrifuge at 1000-2000 rpm for 5 min.
11. Discard supernatant and carefully suspend the cells in 500 μl of PBS.
12. Centrifuge samples at 1000-2000 rpm for 5 min.
13. Discard supernatant and carefully resuspend the cells in 600 μl of FACS buffer containing PI dye.
14. Analyse samples using flow cytometer.

### Knockdown Efficiency

To determine the efficiency in knockdown induction, a dual luciferase assay was performed with tested formulation samples loaded with specific (siLUC), non-specific (NSC4) and unloaded formulations.
1. Seed 6.5×10³ HCES cells per well (on 96-well plate) in 100 μl of DMEM enriched with 10% FBS and grow for 24 hours at standard conditions. Prepare 2 plates to study all formulation samples in 5 replicates each.
2. Transfect the cells with Renilla and Luc2p plasmids using Lipofectamine 2000 following the manufacturer's protocol. To each well add 50 μl of reagent mix containing 0.3 μl of Lipofectamine 2000, 1 ng of Renilla plasmid and 5 ng of Luc2p plasmid diluted in OptiMEM medium to a total volume of 50 μl.
3. Grow the cells for 24 hours at 37° C. with 5% CO₂.
4. Change medium for 90 μl of fresh DMEM per well.
5. Adjust formulation samples to room temperature. Mix 32 μl of each sample with 28 μl of OptiMEM.
6. Prepare control samples with Lipofectamine transfection reagent:
   mix 1.2 μl of Lipofectamine RNAiMAX reagent with 55.8 μl of OptiMEM and incubate for 5 min at room temperature, then add 3 μl of siLUC stock solution and continue incubation for 10 min;
   mix 1.2 μl of Lipofectamine RNAiMAX reagent with 55.8 μl of OptiMEM and incubate for 5 min at room temperature, then add 3 μl of NSC4 stock solution and continue incubation for 10 min;
   mix 1.2 μl of Lipofectamine RNAiMAX reagent with 58.8 μl of OptiMEM and incubate for 15 min at room temperature.
7. Treat cells in 5 replicates by adding 10 μl of loaded or empty formulation or LPF-control per well.
8. Grow cells at standard conditions (37° C. with 5% CO₂).
9. After 48 hours, discard medium and wash wells with PBS.
10. Add 20 μl of Passive Lysis Buffer per well.
11. Incubate plates at room temperature for 15 min on an orbital shaker (900 rpm).
12. Read the luminescence level using Dual-Luciferase Reporter Assay kit and the LUMIstar OPTIMA plate reader, following manufacturer's protocol.

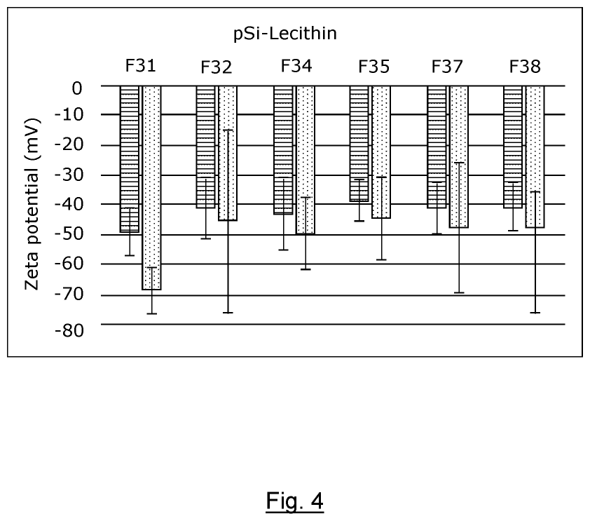

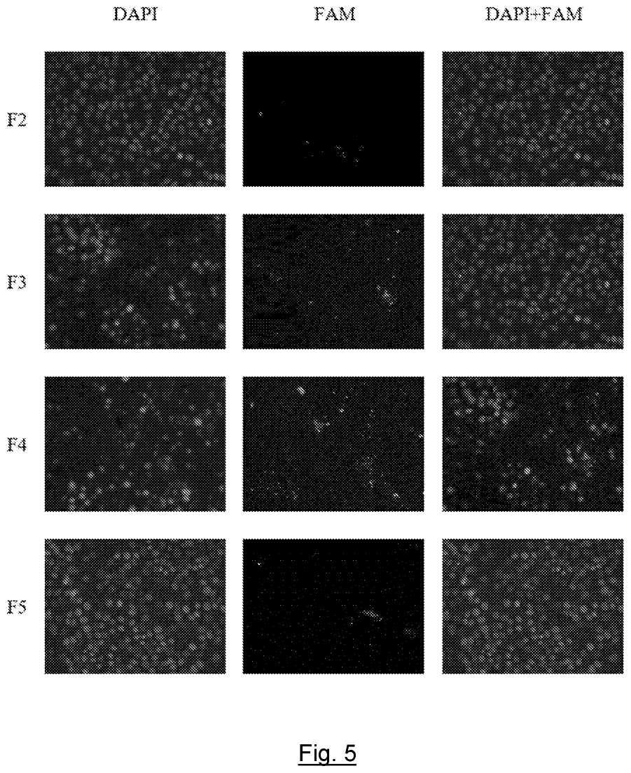

Results are shown in FIG. 5.

### Example 3: Combining siRNA-Loaded Silicon Nanoparticles with Lipofectamine (SIS005-LPF)

1. Aliquot silicon nanoparticle suspension into 6 Eppendorf microtubes so that each contain 20 μg of SiNPs.
2. To above SiNP aliquots add 10 μg of siRNA:
   a. to 2 microtubes add 37 μl of 20 μM siLuc2p=targeted siRNA
   b. to 2 microtubes add 37 μl of 20 μM NSC4=non-specific siRNA
   c. unloaded control: leave the rest 2 microtubes containing SiNPs without siRNA, for preparing empty control
3. Incubate samples for 1 hr at room temperature with agitation, then vortex and place the tubes in the freezer (−20° C.) and keep them for 2-3 hours.
4. Connect samples to freeze-dry system for overnight free drying.
5. Prior to further analysis, dissolve above siRNA-loaded (or unloaded control) SiNP samples in Lipofectamine solution (Lipofectamine RNAiMAX reagent, cat. 13778075, ThermoFisher Scientific) and dilute in nuclease-free water to a total volume of 150 μl, vortex and incubate at RT for 1 hr:

TABLE 4

| Formula | | siRNA | SiNPs | Lipofectamine |
|---|---|---|---|---|
| targeted | SIS005-LPF1 | 10 μg siLuc2p | 20 μg SiNP | 15 μl Lipofectamine |
| non-specific | | 10 μg NSC4 | 20 μg SiNP | 15 μl Lipofectamine |
| empty | | — | 20 μg SiNP | 15 μl Lipofectamine |
| targeted | SIS005-LPF2 | 10 μg siLuc2p | 20 μg SiNP | 30 μl Lipofectamine |
| non-specific | | 10 μg NSC4 | 20 μg SiNP | 30 μl Lipofectamine |
| empty | | — | 20 μg SiNP | 30 μl Lipofectamine |

SIS005-LPF1 and SIS005-LPF2 used Lipofectamine (as lipid), in combination with silicon nanoparticles, at 2 different ratios. LFP1 used 15 μL of 20 μM solution of lipofectamine combined with 20 μg of silicon nanoparticles. LPF2 used a double amount of lipofectamine. The purpose of this experiment was to demonstrate that the combination of lipofectamine with silicon particles could boost transfection promoted by lipofectamine. Results showed silicon nanoparticles improved transfection with lipofectamine.

The siRNA-loading had the greatest impact on surface charge of stearylamine surface treated silicon nanoparticles, inducing a high negative charge. As previous analysis showed, the empty Si-NP loaded with SA and SA+arginine had a positive or nearly neutral surface charge, therefore they attracted anionic siRNA molecules effectively.

### Example 4: The Effect of Loading Ratio on mRNA Entrapment Efficiency

Samples were prepared according to the protocol above for SIS005-DSC613G (Example 2). Each sample comprised a ratio by weight of silicon nanoparticles: dioleoylphosphatidylethanolamine (DOPE):stearylamine:Cholesterol 3β-N-(dimethylaminoethyl) carbamate hydrochloride (DC-cholesterol):glycine of 10:24:4:12:5. The samples were prepared at different ratios of nucleic acid to silicon nanoparticles, as shown in table 5 below.

TABLE 5
ProSilic-DSC613G formulation samples
with varied mRNA loading ratio

| | Loading ratio tested (wt/wt) | | |
|---|---|---|---|
| | silicon | all other | Charge |
| | nanoparticles | components | ratio** |
| Sample | to mRNA | to mRNA * | N/P |
| unloaded U₀ | 0:1 | 0:1 | 0 |
| (control) | | | |
| loaded L_{0.5} | 0.5:1 | 2.75:1 | ~0.625 |
| loaded L₁ | 1:1 | 5.5:1 | ~1.25 |
| loaded L₂ | 2:1 | 11:1 | ~2.5 |
| loaded L₃ | 3:1 | 16.5:1 | ~3.75 |
| loaded L₄ | 4:1 | 22:1 | ~5 |

* This is the ratio by weight of all other components (silicon nanoparticles, lipids and amino acid) to mRNA.
**The approximate charge ratio (known as N/P ratio) is calculated based on the length of the mRNA, the average molecular mass of the RNA (circa 325 Da) and the molecular weight of the cationic lipids (stearylamine:269.5 Da; DC-cholesterol:500.8 Da).

### Example 5: Evaluation of Entrapment of mRNA by Silicon Nanoparticle Formulations Using Gel Electrophoresis

The effect of the loading ratio of silicon nanoparticles to mRNA on mRNA entrapment efficiency was investigated by gel electrophoresis. 1% E-Gel EX pre-cast agarose gel was used. The gel was visualized using the Gel Logic 100 Imaging System (Kodak). The results are shown in FIG. 6.

FIG. 6 shows unloaded mRNA (U0) in line 1. Silicon nanoparticles loaded with mRNA (L0.5, L1, L2, L3, L4, L5, L6, L8) are in lines 2 to 9, in order of increasing ratio of silicon nanoparticles to mRNA, from left to right. An equal amount of mRNA (100 ng) was loaded into each of lines 1 to 9. Invitrogen E-Gel 1 Kb Plus Express DNA Ladder (80 ng) was loaded in line M as a marker. Line 10 was left empty (as a water blank).

Electrophoresis demonstrated that the SIS005-DSC613G formulation of the present invention successfully entraps mRNA, and does so particularly well at higher ratios of silicon nanoparticles to mRNA, such as ratios above 2:1. The control lane (unloaded mRNA, U0) in FIG. 6 shows a single fast-moving band as expected. The band was also visible in line 2 (loaded L0.5) and line 3 (loaded L1). This visible band corresponds to unbound mRNA. The intensity of bands in lines 2 and 3 (particularly in line 3) was lower compared to the control (U0). This indicates that even in samples with a very low ratio of silicon nanoparticles to mRNA, some mRNA is still entrapped by the silicon nanoparticles and is therefore not visible in the band.

Efficiently entrapped mRNA is unable to move through the gel pores and remains in the well (such that no band appears, unlike U0, L0.5 and L1). FIG. 6 shows that samples L2 to L8, with an increased content of silicon nanoparticles compared to lines 1 to 3, successfully entrap mRNA. This is evidenced by the absence of the band seen for U0, L0.5 and L1. This demonstrates that the silicon nanoparticle formulations of the invention are able to successfully entrap mRNA. The results suggest that the optimum loading ratio (the ratio having the greatest entrapment of mRNA, whilst minimising the amount of nanoparticles that are used) is L2, corresponding to ratios of 2:1 (silicon nanoparticles:mRNA) and 11:1 (all other components of the delivery system: mRNA). The same loading ratio has been found to be effective for siRNA loading. This ratio corresponds to an N/P charge ratio of approximately 2.5.

### Example 6: Evaluation of Entrapment Efficiency Using Spectrophotometry

To estimate the entrapment efficiency (EE, expressed as a percentage) of mRNA entrapment, the SIS005-DSC613G samples (U0 to L8) were also centrifuged to separate unbound mRNA. The nucleic acid content in the supernatant fluid was measured by spectrophotometry, and the entrapment efficiency was calculated using the following equation:

$$EE \% = \frac{{OD}_{unloaded} - {OD}_{loaded}}{{OD}_{unloaded}} \times 100 \%$$

OD_{unloaded} is the absorbance for the unloaded mRNA control (U0). OD_{loaded} is the absorbance for each sample L0.5 to L8.

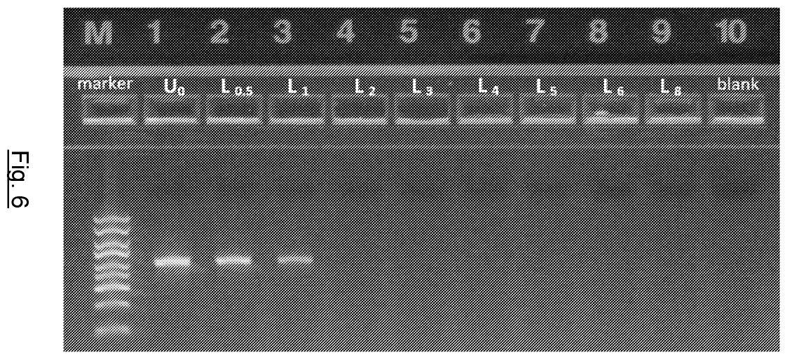

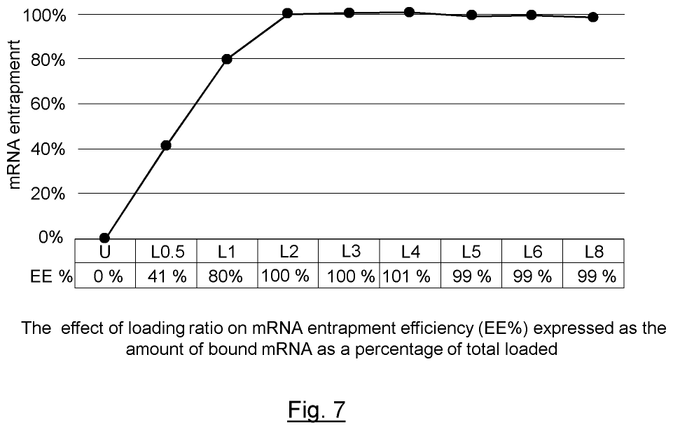

The results are shown in FIG. 7, which confirms the results of the gel electrophoresis experiment. As the ratio of silicon nanoparticles to mRNA increases, the entrapment efficiency increases until it reaches a plateau, levelling off at ratios of silicon to mRNA of greater than 2:1.

### Example 7: Evaluating the Activity of Silicon Nanoparticle Formulations In Vivo

Samples were prepared according to the protocols for SIS005-PS91G and SIS005-DSC613G in Example 2 above. These samples comprise siRNA, silicon nanoparticles, lipids and glycine in the following ratios by weight:

TABLE 6

| | siRNA siLUC/ | silicon | | |
|---|---|---|---|---|
| Sample | NSC4/siGlo | nanoparticles | lipids | glycine |
| SIS005- | 10 | 20 | 80 (72 PC + 8 SA) | 10 |
| PS91G | | | | |
| SIS005- | 10 | 20 | 80 (48 DOPE + 8 | 10 |
| DSC613G | | | SA + 24 DC-Chol) | |

SA is stearylamine, DOPE is dioleoylphosphatidylethanolamine, PC is phosphatidylcholine and DC-Chol is Cholesteryl 3β-N-(dimethylaminoethyl) carbamate hydrochloride.

Samples comprising specific siRNA (siLUC) and non-specific siRNA (NSC4) were prepared. Both siRNAs (siLUC and NSC4) were designed as 21-mers with a central 19 bp duplex region and symmetric dTdT dinucleotide overhangs on each 3′ end. The siRNAs were provided by Eurogentec (Belgium).

To prepare the samples, siRNA dissolved in nuclease-free water was added to an aqueous solution of the nanoparticles and incubated at room temperature for 60 minutes. A ratio of silicon nanoparticles to mRNA of 2:1 was used (as this had been found to be the optimum ratio in gel electrophoresis and spectrophotometry experiments, see above).

### Live Animal Imaging

Animals were used for the following experiments in accordance with the UK Animal Welfare Act, with ethical approval by the Home Office (Scotland) and the Department of Health, Social Services and Public Safety (Northern Ireland). The experiments to assess delivery of fluorescent siRNA (DY-547-labeled siGLO, Dharmacon, UK) to the cornea were performed on wild-type C57BL/6 mice. To assess siRNA bioavailability and silencing activity of formulations, a reporter knock-in mouse line (Krt12+/luc2) was used, with the expression of Firefly luciferase specifically in the cornea epithelium (under the control of the endogenous Krt12 promoter). This animal model was developed on a C57BL/6 background as previously reported and provides a reliable model for in vivo evaluation of siRNA delivery methods using reporter gene expression monitoring. For in vivo imaging, mice were anaesthetized using 1.5-2% isoflurane (Abbott Laboratories Ltd., UK) in a circa 1.5 L/min flow of oxygen. Fluorescence of siGlo was detected with a Xenogen IVIS Spectrum with LivingImage 3.2 software (both Perkin Elmer, UK) using a DsRed filter combination (excitation 535 nm, emission 570 nm) at determined time points following topical application. To measure luciferase reporter gene expression, luciferin (30 mg/mL D-luciferin potassium salt: Gold Biotechnology, USA) mixed 1:1 w/w with Viscotears gel (Novartis, UK) was dropped onto the eye of anaesthetized mouse immediately prior to imaging. Bioluminescence readouts were taken by IVIS Spectrum over a period of approximately 10 min and quantified using LivingImage software after ensuring the signal remained stable within the acquisition time. For signal intensity quantification, a region of interest (ROI) was selected separately for each eye keeping ROI parameters (size and shape) constant throughout experiments. Values are expressed as the right/left eye ratio (RE/LE) using a split body control measurement regime.

In Vivo siRNA Treatment

Experiments were performed using a split body control by comparing the treatment under a test, in one eye, with a negative control in the other eye of the same animal. During treatment, mice were anesthetized as described above. Silicon nanoparticle formulations containing 25 μM siRNA, complexed at a ratio by weight of 2:1 SiNPs-to-mRNA, were prepared and applied topically as a drop to the intact cornea in a total volume of 4 μL per eye. After application, the mouse was kept anesthetized for a further 15 min to allow absorption and maximize uptake. Following treatment, fluorescence and luminescence experiments were performed as described below.

Assessment of siRNA Penetration into the Cornea

To investigate delivery of siRNA to the cornea, an in vivo fluorescence study was performed on wild-type mice using eye drops containing siGlo. The fluorescent siRNA-silicon nanoparticle formulation was applied to the right eye, while the same amount of naked siGlo was applied topically to the left eye of each mouse as a control. Fluorescence live imaging was acquired with IVIS Spectrum at 15 min (i.e. immediately after the treatment procedure) and 3, 6, and 24 h following siGlo application, and signal intensity was normalized to background fluorescence measured prior to treatment (i.e. untreated eyes) and quantified as described earlier. After measurements taken at either 3 h or 24 h, the mice were sacrificed, and the eyes were enucleated, fixed in 4% paraformaldehyde in PBS for 30 min at room temperature, submerged in PolyFreeze (Sigma-Aldrich, UK), and immediately frozen at −80° C. Five-micrometer sections were cut using a cryostat (CM 1850, Leica), mounted on APES-coated slides (3-aminopropyltriethoxysilane, Sigma Aldrich, UK) with DAPI-containing mounting medium (DAPI I, Vysis, USA) and fluorescence was visualized with a AxioScope A1 microscope equipped with a 20×/40× N Archoplan lens on an AxioCam MRc camera (Carl Zeiss, Germany).

Assessment of siRNA-Mediated Gene Silencing

Luciferase reporter mice (n=7) were used to determine the bioavailability of siRNA in the cornea after topical delivery with the silicon nanoparticles of the invention. In a split body control experiment, luciferase-targeting siLuc complexed with the silicon nanoparticle formulation was applied topically as a drop to the intact cornea of the right eye (RE) in anesthetized mice, whereas the left eye (LE) was treated accordingly with NSC4 complexed with the silicon nanoparticle formulation, as a negative control. Treatment was repeated daily for 8 consecutive days, with in vivo ocular luminescence measurements taken approximately 4-5 hours later. The effect of treatment on luciferase reporter gene expression was determined by measurement of luciferase bioluminescent activity (as described above) daily throughout the treatment regimen, and for a further 8 days after cessation of treatment to monitor the wash-out period. Baseline luminescence was defined for each experimental animal by monitoring ocular luciferase activity at 24-hour intervals for 4 days prior to treatment. The relative RE/LE luciferase bioluminescent activity was quantified using the IVIS LivingImage software and plotted as an average value±standard deviation.

### Statistical Analysis

Data are presented as the means±one standard deviation and are representative for at least 3 independent measurements, unless stated otherwise. Statistical significance was assessed with a one or two-way ANOVA followed by the Tukey's HSD post hoc test at 95% confidence level. For in vitro dual luciferase assay, a two-tailed Student's t test was performed for each formulation separately to analyse knockdown level (siLuc vs NSC4 control). For the in vivo luciferase experiment, the statistical analysis was done by comparing the average right/left ratio for all seven mice in the first 4 days before the beginning of the treatment (set as the baseline) with the R/L ratios measured on the following days. Statistical analysis was performed using GraphPad Prism software (GraphPad Software, USA).

### Results

Characteristics of the Silicon-Based siRNA Delivery System

Two replicates of the silicon nanoparticle delivery systems of the invention (SIS005-PS91G and SIS005-DSC613G) were formulated through surface functionalization of silicon with cationic lipids commonly used in nucleic acid delivery, stearylamine and DC-cholesterol, which resulted in hybrid particles with similar hydrodynamic sizes of circa 350 nm and comparative positive zeta potential values. Complexation of cationic silicon nanoparticle formulations with siRNA studied by gel electrophoresis showed full entrapment of nucleic acid with a minimum SiNP-siRNA w/w ratio of 2:1. The percentage of complexed siRNA for varying w/w ratios was determined by spectrophotometry and calculated from the differences of siRNA amounts added to the carrier and the concentration of siRNA in solutions after particle separation. Higher siRNA entrapment efficiency was observed for complexation with nanoparticles containing cationic cholesterol derivative compared to particles functionalized with stearylamine. However, both variants showed siRNA loading capacity in a range of 13-48 nmol per 1 mg of silicon nanoparticles. Following the siRNA loading studies above, a fixed SiNP/siRNA ratio of 2:1 was chosen for all further experiments.

As the physicochemical properties of nanoparticles play an important role in drug delivery, the particle size and surface charge of siRNA-loaded complexes were determined. SIS005-DSC613G did not show significant differences in dimensions or zeta potential when empty and loaded carrier were compared, whereas SIS005-PS91G showed an increased mean particle size and negative surface charge when complexed with siRNA, suggesting an absorption of nucleic acid molecules on the surface of hybrid particles in addition to internal siRNA entrapment (see table below). The formulations considered in this study were compared to the gold standard in gene silencing, Lipofectamine™ RNAiMAX, a commercially available lipid-based carrier specifically designed for siRNA delivery. ZetaSizer analysis of empty and siRNA-loaded RNAiMAX also showed an increase in particle size and an inversion of surface charge from positive to negative values after complexation with nucleic acid.

TABLE 7
Characterization of particle properties

| | | Particle size | Zeta potential |
|---|---|---|---|
| | Sample | [nm] | [mV] |
| SIS005-PS91G | empty | 351 ± 32 | +36.6 ± 1.0 |
| | with siRNA | 462 ± 57 | −39.7 ± 0.6 |
| SIS005-DSC613G | empty | 361 ± 39 | +40.9 ± 0.9 |
| | with siRNA | 397 ± 44 | +34.9 ± 0.7 |
| RNAiMAX | empty | 133 ± 3 | +23.2 ± 12.0 |
| | with siRNA | 212 ± 32 | −57.0 ± 7.1 |

Measurements were undertaken in nuclease-free water. Data represent mean ± standard deviation (n = 3).

### Example 8: Evaluation of In Vitro siRNA Delivery to Corneal Cells

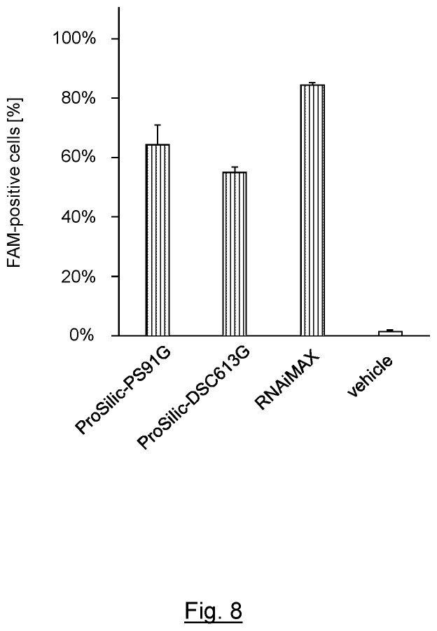

For initial in vitro screening, a human corneal epithelial cell line (HCE-S) was used to evaluate the efficacy of the silicon nanoparticle delivery system of the invention in cellular transfection, along with its potential cytotoxicity. Transfection efficiency was quantified by flow cytometry analysis performed 24 hours after treatment with a fluorescent oligonucleotide duplex loaded to a silicon carrier system, see FIG. 8. SIS005-DSC613G showed 55±2% and SIS005-PS91G showed 65±6% of FAM-positive cells, in comparison to 84±1% observed for the RNAiMAX reagent.

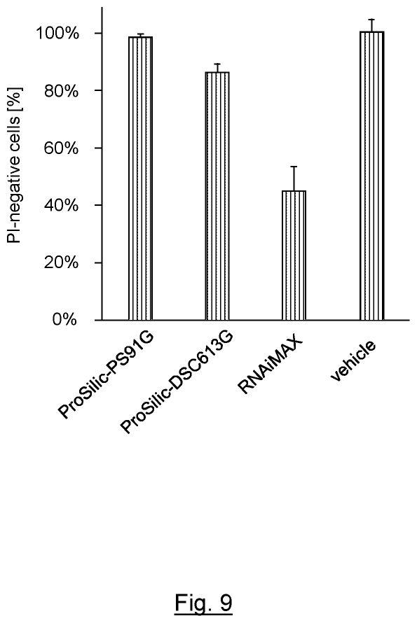

The post-transfection cell viability was assessed based on live/dead staining with propidium iodide (PI), a common indicator of membrane disintegration. This showed that the silicon nanoparticle formulations were well tolerated by corneal epithelial cells in contrast to Lipofectamine, see FIG. 9. Lipofectamine, despite being highly effective for the delivery of exogenous nucleic acids into cells in vitro, is not suitable for clinical applications. Over 50% of cells transfected with Lipofectamine RNAiMAX were shown to have damaged membranes as evidenced by internal staining with the membrane impermeable dye PI whereas more than 86% and 98% intact cells were observed after treatment with the silicon nanoparticle formulations.

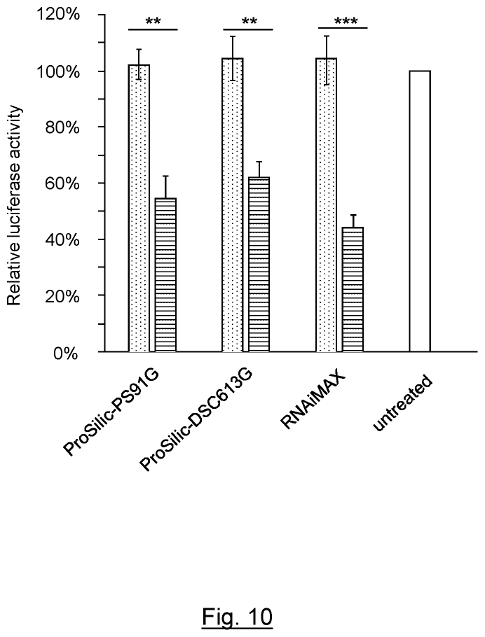

The bioavailability of siRNA was evaluated in gene expression studies using a dual luciferase reporter assay. After treatment of HCE-S cells with 0.1 μM siLuc complexed with the silicon-based delivery system, a knockdown of 46±5% (p<0.01, siLuc vs NSC4 control) and 38±8% (p<0.01) was achieved for SIS005-PS91G and SIS005-DCS613G, respectively, whereas siLuc transfected with RNAiMAX reduced luciferase reporter gene expression by 66±9% (p<0.001). See FIG. 10. Thus, the silicon nanoparticle delivery system demonstrated up to 70% of the potency of a commercial siRNA transfection reagent while being safer and better-tolerated by the cells.

### Example 9: Evaluation of In Vivo Ocular siRNA Delivery by Topical Silicon Nanoparticle Formulations

Following the demonstration of successful siRNA delivery and gene knockdown in vitro, the two silicon nanoparticle formulations (SIS005-PS91G and SIS005-DCS613G) were evaluated in vivo by topical administration to the anterior eye. Firstly, SIS005-PS91G and SIS005-DCS613G were complexed with fluorescent siGlo and applied as an eye drop to wild-type mice following a unilateral procedure with the naked siGlo control being instilled in the opposite eye. The ocular fluorescence was monitored for up to 24 h using an in vivo imaging system. The first measurement was performed 15 min after administration when the mouse was still under anaesthesia after treatment, and the following measurements were repeated at 3, 6, and 24 h post-administration. Although an equal amount of siGlo was applied topically to each eye, the highest fluorescence intensity measured 15 min later was observed for the treatment with SIS005-DSC613G, subtly less for SIS005-PS91G, and two times less for naked siGlo (p<0.05), see FIG. 11. This indicated an increased ocular surface adhesion of formulated drug and an improved residence time for the two silicon nanoparticle formulations in comparison with the naked oligonucleotide. Three hours later, in vivo fluorescence signal decreased 3-fold in siGlo-SIS005-DSC613G-treated eyes, whereas it had returned to baseline levels for siGlo-SIS005-PS91G and unformulated siGlo eye drops due to active ocular clearance mechanisms. Although a further gradual decrease of in vivo signal intensity was observed, fluorescence in the eyes treated with siGlo-SIS005-DSC613G persisted for up to 24 h and was significantly higher than in eyes treated with the unformulated naked siGlo at all the time points (p<0.01 at 3 h and 6 h, and p<0.05 at 24 h). This suggests effective uptake of topically administered siRNA drug formulated with the silicon nanoparticles of the invention. To verify nanoparticle permeation into the tissue, distribution of the siRNA throughout the cornea layers was investigated by fluorescence microscopy of post-treatment corneal sections. Red siGlo fluorescence was detected throughout all corneal layers in all treated sections of eyes collected 3 hours after eye drops application, whereas no fluorescence above background was observed in the naked siGlo control. Twenty-four hours after the administration of siRNA formulations, the fluorescence was only observed in the cornea sections treated with SIS005-DSC613G.

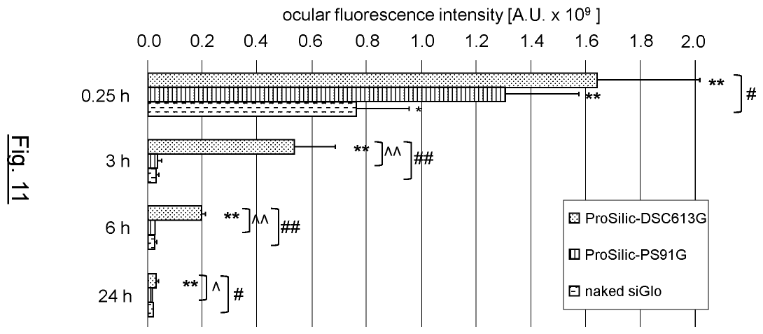

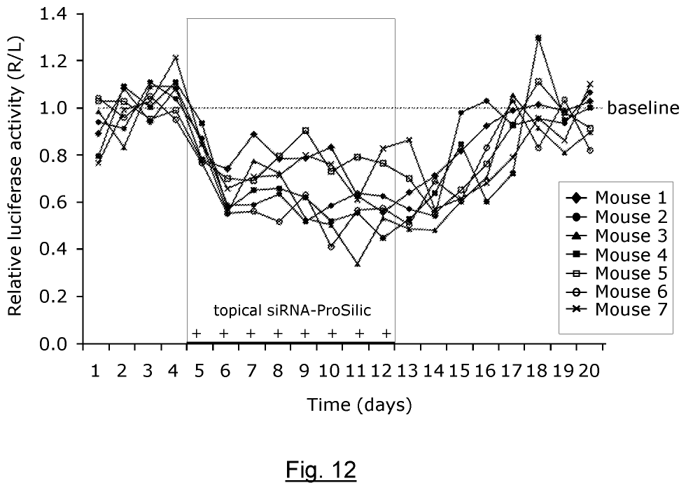

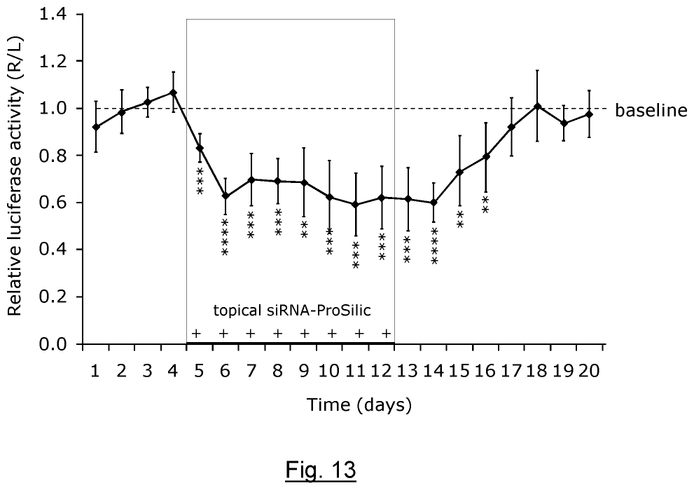

Following the in vivo uptake studies, siRNA delivery with SIS005-DSC613G was further investigated in functional assays using a murine reporter model with luciferase expression confined exclusively to the corneal epithelium. Prior to in vivo treatment, basal corneal luciferase activity in reporter mice was quantified every 24 h for 4 days to confirm a consistent right-to-left ratio for a split body control experiment. SIS005-DSC613G complexed with siLuc or control siRNA was applied topically as eye drops to opposite eyes of the same animal 8 times at daily intervals, and corneal luciferase expression was evaluated every day by live animal imaging throughout treatment regimen and over following 8 days. A reduced luciferase expression was observed within 24 h of treatment initiation, and a maximal inhibition (41%±13, p<0.001) was achieved at day 11. A significant gene silencing effect persisted throughout the entire treatment regimen and continued for 4 days after termination of treatment. As expected, the reduced ocular bioluminescence level gradually returned to baseline after treatment withdrawal which indicated a successful recovery from gene repression. See FIGS. 12 and 13 for these results. Importantly, gross examination of treated eyes and daily visual inspection of animals following topical treatment revealed no adverse effects from the eye drops, suggesting that the silicon nanoparticle formulation was well-tolerated in vivo.

### Increased Nucleic Acid Binding Efficiency of Si-Containing Formulation Compared to Known Liposomes

Sample formulations comprising silicon nanoparticles were prepared according to the protocol above. Also prepared were formulations without silicon nanoparticles. The formulations have the compositions shown in the table below.

TABLE 8
Composition of Si nanoparticle-containing formulations and composition
of liposome formulations without Si nanoparticles*

| | Unloaded mRNA | DSC613G | | DS61G | | DS64G | | D6G | |
|---|---|---|---|---|---|---|---|---|---|
| | (control) | LBC | LL | LBC | LL | LBC | LL | LBC | LL |
| mRNA/hsDNA | 50 | 50 | 50 | 50 | 50 | 50 | 50 | 50 | 50 |
| SiNP | — | 100 | — | 100 | — | 100 | — | 100 | — |
| DOPE | — | 240 | 240 | 240 | 240 | 240 | 240 | 240 | 240 |
| SA | — | 40 | 40 | 40 | 40 | 160 | 160 | — | — |
| DC-Chol | — | 120 | 120 | — | — | — | — | — | — |
| Gly | — | 50 | 50 | 50 | 50 | 50 | 50 | 50 | 50 |

*SiNP refers to Si nanoparticles; DOPE refers to dioleoylphosphatidylethanolamine; SA refers to stearylamine; DC-Chol refers to Cholesteryl 3β-N-(dimethylaminoethyl)carbamate hydrochloride; and Gly refers to glycine. LBC refers to the Si-containing formulation, whereas LL refers to liposomal formulations without Si.

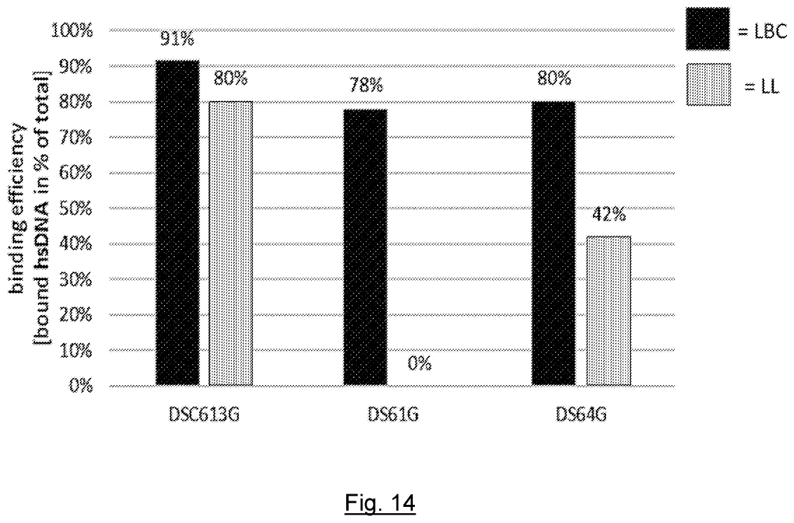

The nucleic acid binding efficiency for hsDNA was investigated for each of these compositions. The results are shown in FIG. 14. As FIG. 14 shows, the binding efficiency observed for the Si nanoparticle-containing formulations showed improvement over the liposome formulations not comprising silicon nanoparticles.

### Example 10: Further Investigation Using Biocourier Formulations

“Biocourier” as used in these examples refers to vector compositions according to the invention, comprising silicon, lipid and optional other ingredients in accordance with the invention, onto which a nucleic acid may be loaded.

### Preparation of Starting Materials

a) Non activated Boron Doped Silicon wafer: Obtained from BS Silicon and manually milled
b) Non Activated AE Silicon: as provided by American Elements, 30 nm average diameter, porous
c) Activated AE Silicon: American Elements, 30 nm average diameter, porous. 1 g of material suspended in 50 mL of MeOH, then subjected to slow evaporation process under fume hood.
d) Activated BS Silicon: Porous. 1 g of material suspended in 50 mL of MeOH, then subjected to slow evaporation process under fume hood
e) DOTAP-CI Solution: DOTAP was solubilized in methanol at the concentration 5 mg/mL. In particular, 50 mg of DOTAP were solubilized in 10 ml of methanol and sonicated until fully solubilised.
f) DOPE Solution: DOPE was solubilized in methanol at the concentration 5 mg/mL. In particular, 60 mg of DOPE were solubilized in 12 ml of methanol and sonicated until fully solubilised.
g) mPEG2000-DSPE Solution: mPEG2000-DSPE was solubilized in methanol at the concentration 5 mg/ml. In particular, 40 mg of mPEG2000-DSPE were solubilized in 8 ml of methanol and sonicated until fully solubilised.
h) Trehalose (THR): as provided by Sigma Aldrich, powder.
i) Glycine (GLY): as provided by Sigma Aldrich, powder.
j) SiNPs AE+GLY+THR Solution: activated SiNPs (AE), Glycine and Trehalose were suspended in nuclease-free water. In particular, 50 mg of activated SiNPs, 50 mg of Trehalose and 25 mg of Glycine were suspended in 50 mL of nuclease-free water and sonicated for 60 minutes.
k) SiNPs BS+GLY+THR Solution: activated SiNPs (BS), Glycine and Trehalose were suspended in nuclease-free water. In particular, 50 mg of activated SiNPs, 50 mg of Trehalose and 25 mg of Glycine were suspended in 50 mL of nuclease-free water and sonicated for 60 minutes.
   Preparation of pDNA-Biocourier Aliquots for Immunization Studies
   Selection of Biocourier Type and Calculation of Ratio for Optimal pDNA Complexation

Biocourier formulation type 1, prepared using American Elements (AE) Silicon (porous, activated, Average particle size<100 nm)−ingredients and ratio:

TABLE 9
Composition of Biocourier type 1

| | | | | | | Activated | | | |
|---|---|---|---|---|---|---|---|---|---|
| Lipids | | | | | | SiNPs AE | | | Nuclease-free |
| DOTAP | | DOPE | | DSPE | | (≤100 nm) | Glycine | Trehalose | water |
| 7.25 | mg | 7.30 | mg | 1.45 | mg | 1 mg | 0.5 mg | 1 mg | Up to 10 mL |

| | | | | | | | |
|---|---|---|---|---|---|---|---|
| 1.45 | mL | 1.46 | mL | 0.29 | mL | 1 mL | Up to 10 mL |

Ratio Lipids: Si (before extrusion)=16:1
16 mg total lipids/10 mL Biocourier, 1.6 mg/mL of lipids and 0.1 mg/mL silicon, equivalent to:
Biocourier stock solution=1.6 μg/μL
pDNA stock solution=2 μg/μL

Each vial must contain:
80 μg of pDNA
400 μL of total volume
80 μg pDNA equivalent to 40 μL-remaining volume=400 μL (total volume)−40 μL (pDNA volume)=360 μL
360 μL can contain up to 1.6 μg/μL×360 μL=576 μg Biocourier
Ratio Biocourier: pDNA=576:80=7.2:1
Using this format, 50 μL (single dose/mouse) of SiSaf Biocourier/pDNA complex will contain:
10 μg pDNA and 72 μg of Biocourier

All samples are provided as extruded samples, suspended in nuclease-free water. Extrusion is considered the gold-standard practice for preparing injectable samples, and refers to passing the suspended particles through a filter membrane multiple times in order to ensure that the particles are not unacceptably clumped.

Biocourier Injectable i.d./i.m. was extruded by 400 nm and 100 nm polycarbonate membranes, ready to be complexed with the relevant amount of pDNA.

### Preparation of Described Biocourier Formulation Type 1: Liquid Form

### A) Lipid Film Preparation

1) Transfer the amount of Lipids, as given in Table 9 above from the stock solution into a clean glass round bottom flask and mix.
2) Carefully evaporate the solvent using a rotary evaporator using a water bath at 40° C. and vacuum.

### B) Rehydration of the Film

3) Add the Si-NPs+GLY+THR Solution to the film and if necessary, adjust the final volume to 10 mL, using nuclease free water.
4) Cover the flask with parafilm and rehydrate the film, agitating the flask in a water bath (60° C.) for 5 minutes.
5) Split 1 mL of the samples into RNA-free Eppendorf tubes, 10 Eppendorfs in total. Store the suspension in the fridge.

### C) Extrusion Process

6) The suspension should be passed through a membrane filter of pore sizes 0.4 μm and 0.1 μm, 10 cycles through 0.4 μm and then 10 cycles through 0.1 μm at 60° C. Store the suspension in the fridge after the extrusion is completed.
   D) Loading of the Biocourier with pDNA
7) Thaw the pDNA sample stored at −40° C. and leave the tube under a fume hood until it reaches room temperature (expected time period 10 min).
8) Equilibrate the extruded Biocourier sample by transferring the sample from the fridge under a fume hood, at room temperature (expected time period 6 min).
9) Load the relevant amount of Biocourier with pDNA into a sterile Eppendorf tube. In particular, mix 360 μL of Biocourier with 40 μL of pDNA solution. Seal the Eppendorf.
10) Gently vortex the Eppendorf for 15 seconds and leave the sealed sample equilibrating for 30 min at room temperature. Transfer the sample into a refrigerator until usage. Follow provided instruction before injecting.

Prepare 4 aliquots as described.

### Preparation of Described Biocourier Formulation Type 1: Freeze-Dried Form

### A) Lipid Film Preparation

1) Transfer the amount of lipids, given in Table 9 above, from the stock solution into a clean glass round bottom flask and mix.
2) Carefully evaporate the solvent using a rotary evaporator using a water bath at 40° C. and vacuum.

### B) Rehydration of the Film

3) Add the Si-NPs+GLY+THR Solution to the film and if necessary, adjust the final volume to 10 mL, using nuclease free water.
4) Cover the flask with parafilm and rehydrate the film, agitating the flask in a water bath (60° C.) for 5 minutes.
5) Split 1 mL of the samples in RNA-free Eppendorf tubes, 10 Eppendorfs in total. Store the suspension in the fridge.

### C) Extrusion Process

6) The suspension should be passed through a membrane filter of pore sizes 0.4 μm and 0.1 μm, 10 cycles through 0.4 μm and then 10 cycles through 0.1 μm at 60° C. Store the suspension in the fridge after the extrusion is completed.
   D) Loading of the Biocourier with pDNA
7) Thaw the pDNA sample stored at −40° C. and leave the tube under a fume hood until it reaches room temperature (expected time period 10 min).
8) Equilibrate the extruded Biocourier sample by transferring the sample from the fridge to a fume hood, at room temperature (expected time period 6 min).
9) Load the relevant amount of Biocourier with pDNA into a sterile Eppendorf. In particular, mix 360 μL of Biocourier with 40 μL of pDNA solution. Seal the Eppendorf.
10) Gently vortex the Eppendorf for 15 seconds and leave the sealed sample equilibrating for 30 min at room temperature. Transfer the sample to a refrigerator for 3 hrs.
11) Move samples to −40° C. for freezing them (5 hrs).
12) Activate freeze drier and allow thermostatic chamber to reach-40° C. Move the samples to the freeze drier and activate the vacuum.
13) Freeze dry samples overnight.
14) Seal obtained samples and move them to the refrigerator.

Prepare 4 aliquots as described.

Biocourier formulation type 2, prepared using boron-doped silicon (porous, activated-ingredients and ratio:

TABLE 10
Composition of Biocourier type 2

| | | | | | | Activated | | | |
|---|---|---|---|---|---|---|---|---|---|
| Lipids | | | | | | SiNPs BS | | | Nuclease-free |
| DOTAP | | DOPE | | DSPE | | (≤100 nm) | Glycine | Trehalose | water |
| 7.25 | mg | 7.30 | mg | 1.45 | mg | 1 mg | 0.5 mg | 1 mg | Up to 10 mL |

| | | | | | | | |
|---|---|---|---|---|---|---|---|
| 1.45 | mL | 1.46 | mL | 0.29 | mL | 1 mL | Up to 10 mL |

Ratio Lipids: Si (before extrusion)=16:1
16 mg total lipids/10 mL Biocourier, 1.6 mg/mL of lipids and 0.1 mg/mL Silicon, equivalent to:
Biocourier stock solution=1.6 μg/μL
pDNA stock solution=2 μg/μL
each vial has to contain:
80 μg of pDNA
400 μL of total volume
80 μg pDNA equivalent to 40 μL−remaining volume=400 μL (total volume)−40 μL (pDNA volume)=360 μL
360 μL can contain up to 1.6 μg/μL×360 μL=576 μg Biocourier
Ratio Biocourier: pDNA=576:80=7.2:1
Using this format, 50 μL (single dose/mouse) of Biocourier/pDNA complex will contain:
10 μg pDNA and 72 μg of Biocourier

All samples are provided as extruded samples, suspended in nuclease-free water. Extrusion is considered the gold standard practice for preparing injectable samples.

Biocourier Injectable i.d./i.m. extruded through 400 nm and 100 nm polycarbonate membranes. Then Biocourier extruded samples to be complexed with the relevant amount of pDNA.

### Preparation of Described Biocourier Formulation Type 2: Liquid Form

### A) Lipid Film Preparation

1) Transfer the amount of Lipids, given in Table 10 above, from the stock solution into a clean glass round bottom flask and mix.
2) Carefully evaporate the solvent using a rotary evaporator using a water bath at 40° C. and vacuum.

### B) Rehydration of the Film

3) Add the Si-NPs+GLY+THR Solution to the film and if necessary, adjust the final volume to 10 mL, using nuclease free water.
4) Cover the flask with parafilm and rehydrate the film, agitating the flask in a water bath (60° C.) for 5 minutes.
5) Split 1 mL of the samples into RNA-free Eppendorf tubes, 10 Eppendorf in total. Store the suspension in the fridge.

### C) Extrusion Process

6) The suspension should be passed through a membrane filter of pore sizes 0.4 μm and 0.1 μm, 10 cycles through 0.4 μm and then 10 cycles through 0.1 μm at 60° C. Store the suspension in the fridge after the extrusion is completed.
   D) Loading of the Biocourier with pDNA
7) Thaw the pDNA sample stored at −40° C. and leave the tube under a fume hood until it reaches room temperature (expected time period 10 min).
8) Equilibrate the extruded Biocourier sample by transferring the sample from the fridge to a fume hood, at room temperature (expected time period 6 min).
9) Load the relevant amount of Biocourier with pDNA into a sterile Eppendorf. In particular, mix 360 μL of Biocourier with 40 μL of pDNA solution. Seal the Eppendorf.
10) Gently vortex the Eppendorf for 15 seconds and leave the sealed sample equilibrating for 30 min at room temperature. Transfer the sample in a refrigerator until usage. Follow provided instruction before injecting.

Prepare 4 aliquots as described.

### Preparation of Described Bio-Courier Formulation Type 2: Freeze-Dried Form

### A) Film Preparation

1) Transfer the amount of lipids, as describe in Table 3 above, from the stock solution in a clean glass round bottom flask, and mix.
2) Carefully evaporate the solvent using a rotary evaporator and using a water bath at 40° C., under vacuum.

### B) Rehydration of the Film

3) Add the Si-NPs+GLY+THR Solution onto the film and, if necessary, adjust the final volume to 10 mL, using nuclease free water.
4) Cover the flask with parafilm and rehydrate the film, agitating the flask in a water bath (60° C.) for 5 minutes.
5) Split 1 mL of the samples in an RNA-free Eppendorf, 10 Eppendorf in total. Store the suspension in the fridge.

### C) Extrusion Process

6) The suspension should be passed through a membrane filter of pore sizes 0.4 μm and 0.1 μm: 10 cycles through 0.4 μm; and then 10 cycles through 0.1 μm, at 60° C. Store the suspension in the fridge after the extrusion is completed.
   D) Loading of the Biocourier with pDNA
7) Thaw the pDNA sample stored at −40° C.; leave the tube under a fume hood until it reaches room temperature (expected time period: 10 min).
8) Equilibrate the extruded Biocourier sample, by transferring the sample from the fridge to under a fume hood, at room temperature (expected time period: 6 min).
9) Load the relevant amount of Biocourier with pDNA into a sterile Eppendorf. In particular, mix 360 μL of Biocourier with 40 μL of pDNA solution. Seal the Eppendorf.
10) Gently vortex the Eppendorf for 15 seconds and leave the sealed sample equilibrating for 30 min at room temperature. Transfer the sample to a refrigerator for 3 hrs.
11) Move samples to −40° C. for freezing them (5 hrs).
12) Activate freeze drier and allow thermostatic chamber to reach −40° C. Move the samples to the freeze drier and activate the vacuum.
13) Freeze dry samples overnight.
14) Seal obtained samples and move them to a refrigerator.

Prepare 4 aliquots as described.

### Reconstitution of Samples and General Recommendations for Handling

### Instruction for Freeze Dried-Powdered Sample Reconstitution

Transfer an Eppendorf tube to a fume hood and re-suspend the contents of the Eppendorf, using 400 μL nuclease free water.

Re-suspension is intended to be performed at room temperature under a fume hood and using appropriate sterile protective equipment, thus avoiding any cross contamination of the contents.

After re-suspension, close the Eppendorf tube; gently vortex for 15 seconds; and leave the sealed sample equilibrating for 30 min at room temperature.

Transfer the sample to a refrigerated container and bring it to room temperature a few minutes before administration. Before sampling, gently invert the Eppendorf twice.

Rehydrate only vials intended to be used/administered on same day.

### Instruction for Liquid Samples

Transfer the Eppendorf to a fume hood. Equilibrate for 3 min at room temperature and gently vortex for 15 seconds; leave the sealed sample equilibrating for 30 min at room temperature.

Transfer the sample to a refrigerated container and bring it to room temperature a few minutes before administration. Before sampling, gently invert the Eppendorf twice.

### Example 11: Considerations Regarding Extrusion, and how this Process Step May Alter the Optimum Starting Ratios of Ingredients

Extrusion can generate loss of silicon particles with respect to the initial amount used. For present purposes, a final silicon content after extrusion of about 2 mg/L, equivalent to 2 μg/mL, was sought.

Generally, to produce 1 mL of finished vector composition meeting that requirement, it is recommended to begin with 1.6 mg of the lipid component, 0.1 mg trehalose (if present), 0.05 mg glycine (if present) and 2 μg silicon.

For simplicity, when reference is made herein to the ratio of Biocourier: nucleic acid, this is the molar ratio between the lipid component of the Biocourier and the nucleic acid.

Thus, exemplary compositions are given in Table 11 below.

TABLE 11
Example compositions

| No | ID | Samples of Si-NP | Ratio of components | siRNA loaded per sample |
|---|---|---|---|---|
| 1 | F1 | 75 μg Si | 3.75:0:0 | 150 μg siLuc2p |
| 2 | F2 | 75 μg Si:200 μg PC | 3.75:10:0 | 150 μg siLuc2p |
| 3 | F3 | 75 μg Si:200 μg PC:20 μg Gly | 3.75:10:1 | 150 μg siLuc2p |
| 4 | F4 | 75 μg Si:200 μg PC:20 μg Arg | 3.75:10:1 | 150 μg siLuc2p |
| 5 | F5 | 75 μg Si:200 μg PC:20 μg His | 3.75:10:1 | 150 μg siLuc2p |

### Example 12: Assessment of Zeta Potential

Formulations were produced having ingredients as shown in table 12. The measured zeta potential of those formulations are shown in table 13. The influence of ingredients on zeta potential can be assessed and the change in zeta potential on loading with RNA can be used as a simple quality control marker as evidence of successful complexing.

TABLE 12
Tested compositions

| | | | | Si to |
|---|---|---|---|---|
| Formulation sample | | | siRNA loaded | siRNA |
| No | ID | ratio | per sample | ratio |
| 1 | F01 | 75 μg Si:200 μg PC | 150 μg siLuc2p | 1:2 |
| 2 | F02 | 150 μg Si:200 μg PC | 150 μg siLuc2p | 1:1 |
| 3 | F04 | 75 μg Si:200 μg PC:20 μg Arg | 150 μg siLuc2p | 1:2 |
| 4 | F05 | 150 μg Si:200 μg PC:20 μg Arg | 150 μg siLuc2p | 1:1 |
| 5 | F07 | 75 μg Si:200 μg PC:40 μg Arg | 150 μg siLuc2p | 1:2 |
| 6 | F08 | 150 μg Si:200 μg PC:40 μg Arg | 150 μg siLuc2p | 1:1 |
| 7 | F11 | 75 μg Si:200 μg SA | 150 μg siLuc2p | 1:2 |
| 8 | F12 | 150 μg Si:200 μg SA | 150 μg siLuc2p | 1:1 |
| 9 | F14 | 75 μg Si:200 μg SA:20 μg Arg | 150 μg siLuc2p | 1:2 |
| 10 | F15 | 150 μg Si:200 μg SA:20 μg Arg | 150 μg siLuc2p | 1:1 |
| 11 | F17 | 75 μg Si:200 μg SA:40 μg Arg | 150 μg siLuc2p | 1:2 |
| 12 | F18 | 150 μg Si:200 μg SA:40 μg Arg | 150 μg siLuc2p | 1:1 |
| 13 | F21 | 75 μg Si:200 μg PE | 150 μg siLuc2p | 1:2 |
| 14 | F22 | 150 μg Si:200 μg PE | 150 μg siLuc2p | 1:1 |
| 15 | F24 | 75 μg Si:200 μg PE:20 μg Arg | 150 μg siLuc2p | 1:2 |
| 16 | F25 | 150 μg Si:200 μg PE:20 μg Arg | 150 μg siLuc2p | 1:1 |
| 17 | F27 | 75 μg Si:200 μg PE:40 μg Arg | 150 μg siLuc2p | 1:2 |
| 18 | F28 | 150 μg Si:200 μg PE:40 μg Arg | 150 μg siLuc2p | 1:1 |
| 19 | F31 | 75 μg Si:200 μg Lecithin | 150 μg siLuc2p | 1:2 |
| 20 | F32 | 150 μg Si:200 μg Lecithin | 150 μg siLuc2p | 1:1 |
| 21 | F34 | 75 μg Si:200 μg Lec:20 μg Arg | 150 μg siLuc2p | 1:2 |
| 22 | F35 | 150 μg Si:200 μg Lec:20 μg Arg | 150 μg siLuc2p | 1:1 |
| 23 | F37 | 75 μg Si:200 μg Lec:40 μg Arg | 150 μg siLuc2p | 1:2 |
| 24 | F38 | 150 μg Si:200 μg Lec:40 μg Arg | 150 μg siLuc2p | 1:1 |

TABLE 13
Results of zeta potential measurement for unloaded and siRNA-loaded formulations

| Formulation sample | | | Zeta potential | |
|---|---|---|---|---|
| No. | ID | ratio | unloaded | loaded with siRNA |
| 1 | F01 | 75 μg Si:200 μg PC | −30.4 mV ± 7.7 | −50.1 mV ± 9.4 |
| 2 | F02 | 150 μg Si:200 μg PC | −25.4 mV ± 8.0 | −41.7 mV ± 6.6 |
| 3 | F04 | 75 μg Si:200 μg PC:20 μg Arg | −31.4 mV ± 8.2 | −49.6 mV ± 18.6 |
| 4 | F05 | 150 μg Si:200 μg PC:20 μg Arg | −28.8 mV ± 7.8 | −35.3 mV ± 10.0 |
| 5 | F07 | 75 μg Si:200 μg PC:40 μg Arg | −34.6 mV ± 5.7 | −45.8 mV ± 13.9 |
| 6 | F08 | 150 μg Si:200 μg PC:40 μg Arg | −31.4 mV ± 7.0 | −34.3 mV ± 11.3 |
| 7 | F11 | 75 μg Si:200 μg SA | +23.0 mV ± 8.9 | −34.4 mV ± 11.2 |
| 8 | F12 | 150 μg Si:200 μg SA | +13.5 mV ± 7.9 | −35.9 mV ± 14.4 |
| 9 | F14 | 75 μg Si:200 μg SA:20 μg Arg | +14.8 mV ± 7.6 | −31.3 mV ± 9.1 |
| 10 | F15 | 150 μg Si:200 μg SA:20 μg Arg | +1.65 mV ± 4.9 | −30.3 mV ± 7.4 |
| 11 | F17 | 75 μg Si:200 μg SA:40 μg Arg | +11.6 mV ± 6.3 | −30.2 mV ± 8.1 |
| 12 | F18 | 150 μg Si:200 μg SA:40 μg Arg | −4.33 mV ± 6.1 | −35.4 mV ± 8.3 |
| 13 | F21 | 75 μg Si:200 μg PE | −34.8 mV ± 8.2 | −37.3 mV ± 6.5 |
| 14 | F22 | 150 μg Si:200 μg PE | −31.5 mV ± 9.6 | −30.8 mV ± 10.1 |
| 15 | F24 | 75 μg Si:200 μg PE:20 μg Arg | −43.5 mV ± 9.1 | −38.8 mV ± 9.9 |
| 16 | F25 | 150 μg Si:200 μg PE:20 μg Arg | −38.7 mV ± 8.5 | −38.3 mV ± 7.8 |
| 17 | F27 | 75 μg Si:200 μg PE:40 μg Arg | −45.7 mV ± 8.3 | −46.9 mV ± 8.9 |
| 18 | F28 | 150 μg Si:200 μg PE:40 μg Arg | −36.7 mV ± 9.6 | −41.7 mV ± 11.2 |
| 19 | F31 | 75 μg Si:200 μg Lecithin | −48.7 mV ± 7.7 | −68.2 mV ± 7.7 |
| 20 | F32 | 150 μg Si:200 μg Lecithin | −40.7 mV ± 10.4 | −45.0 mV ± 30.3 |
| 21 | F34 | 75 μg Si:200 μg Lec:20 μg Arg | −42.9 mV ± 11.8 | −49.2 mV ± 12.1 |
| 22 | F35 | 150 μg Si:200 μg Lec:20 μg Arg | −38.2 mV ± 7.0 | −44.4 mV ± 13.7 |
| 23 | F37 | 75 μg Si:200 μg Lec:40 μg Arg | −40.8 mV ± 8.8 | −47.3 mV ± 21.5 |
| 24 | F38 | 150 μg Si:200 μg Lec:40 μg Arg | −40.4 mV ± 8.1 | −47.7 mV ± 13.2 |

### Example 13: Demonstration of Benefits of Silicon on Nucleic Acid Binding Efficiency and on the Complex's Stability

A series of Biocourier formulations (produced as described in the preceding Examples) were screened to assess whether the presence of silicon exerts a beneficial effect on nucleic acid binding efficiency and stability, in comparison with lipid nanoparticle compositions formulated with the same components, and in the same manner, as the respective Biocourier composition, but without the silicon-containing particles.

In particular, a study was performed using Biocourier formulations DSC613G, DS61G, DS6G and D6G, with crude extract of herring sperm (hsDNA) as an example of DNA, and a yeast mRNA as an example of mRNA, to assess the impact of silicon particles on nucleic acid binding efficiency and stability.

TABLE 14
Concentration of components in samples (μg/mL): various Biocouriers and respective
liposome formulations loaded with mRNA (or hsDNA in a repeat study) at fixed L2 ratio

| | Unloaded | DSC613G | | DS61G | | DS64G | | D6G | |
|---|---|---|---|---|---|---|---|---|---|
| | mRNA | Loaded | loaded | loaded | loaded | loaded | Loaded | loaded | loaded |
| | (control) | Biocourier | liposome | Biocourier | liposome | Biocourier | liposome | Biocourier | liposome |
| mRNA or | 50 | 50 | 50 | 50 | 50 | 50 | 50 | 50 | 50 |
| hsDNA | | | | | | | | | |
| SiNP | — | 100 | — | 100 | — | 100 | — | 100 | — |
| DOPE | — | 240 | 240 | 240 | 240 | 240 | 240 | 240 | 240 |
| SA | — | 40 | 40 | 40 | 40 | 160 | 160 | — | — |
| DC-Chol | — | 120 | 120 | — | — | — | — | — | — |
| Gly | — | 50 | 50 | 50 | 50 | 50 | 50 | 50 | 50 |

| | | | |
|---|---|---|---|
| standard lipid base | without DC-Chol | without DC-Chol | without |

| | | |
|---|---|---|
| (amount of DOPE and SA | (amount of SA increased | DC-Chol and SA |
| unchanged) | to recompense the charge) | (DOPE only) |

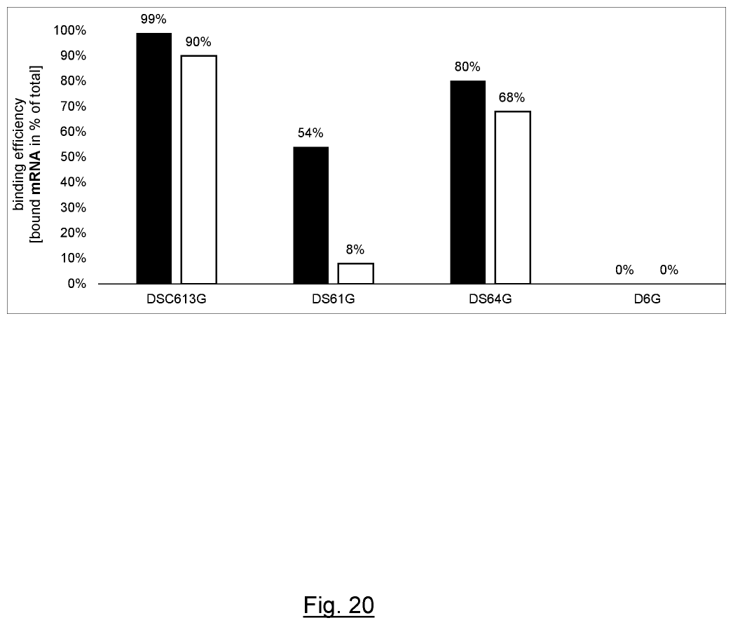

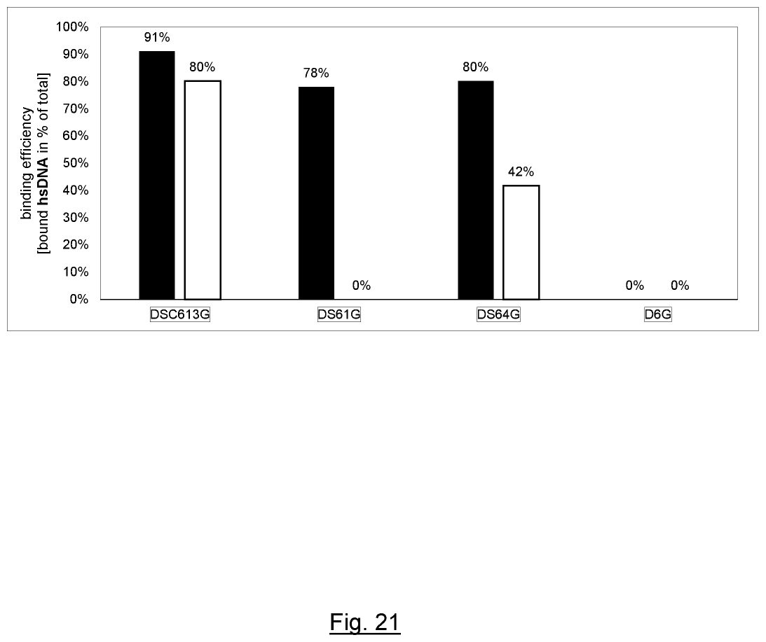

Results are shown in FIGS. 20 and 21. The binding efficiency (and thus, it is inferred, the stability) of the nucleic acid (hsDNA or mRNA) is evidently improved when silicon particles are present. Thus, as demonstrated in these Figures, it was found that nucleic acid binding and stability are superior for formulations containing silicon particles as opposed to corresponding formulations made without the silicon particles.

### Example 14-Stability of Dasher GFP mRNA Complexed with Si

### Materials

mRNA

A solution of 200 μg (200 μL) of Dasher GFP mRNA in nuclease free water (1 mg/mL) was obtained from Aldevron (4055 41st Avenue South Fargo, North Dakota 58104, USA). The mRNA was further diluted with 200 μL of nuclease free water to reach a final concentration of 0.5 mg/mL then aliquoted into 50 μL aliquots in 0.2 mL PCR tubes.

### Bovine Serum

Bovine serum was obtained in frozen liquid form, from Merck (The Old Brickyard, New Rd, Gillingham, Dorset, SP8 4XT). Upon receipt, the serum was defrosted and from it were prepared aliquots in sterile 15 mL and 50 mL falcon tubes, which were then stored at −20° C. 1 mL of the serum was mixed with 4 mL of nuclease free water, to give 5 mL of 20% serum. From this sample, 4% serum was prepared by mixing 1 mL of the 20% serum with 4 mL of nuclease free water. The 20% and 4% serum samples were then aliquoted into 1 mL aliquots in nuclease 2 mL PCR tubes (nuclease and protease free) and stored at −20° C. until being used for the assay.

### SIS0012. SIS0013 and Lipid Nanoparticle

SIS0012 (undoped Si) and SIS0013 (boron-doped Si; 5×10¹⁸ boron atoms/cm³) samples were provided according to the protocol above.

Also provided was a lipid nanoparticle sample containing all components, except silicon, of SIS0012 and SIS0013.

The mRNA was loaded on SIS0012, SIS0013 and the lipid nanoparticle sample according to the protocol above. The ratio by weight of mRNA to each of SIS0012, SIS0013 and LNP, was 1:12.

### Experimental Procedure

Each of the following samples were added to 8 0.2 mL PCR tubes (30 μL per tube):
mRNA-SIS0012 complex
mRNA-SIS0013 complex
mRNA-lipid nanoparticle (excluding silicon) complex (“mRNA-X”)
a control sample of mRNA in nuclease free water

All samples were mixed with bovine serum, incubated at 37° C. and then analysed by gel electrophoresis, according to the following procedure:

30 μL of bovine serum (4%) was added to each tube, which was then mixed thoroughly by pipetting up and down 4-5 times. The cap was tightly closed. The tube was over-sealed with parafilm and placed inside a microtube rack in a water bath at 37° C. As a no-serum control, one tube was mixed only with 20 μL of nuclease free water.

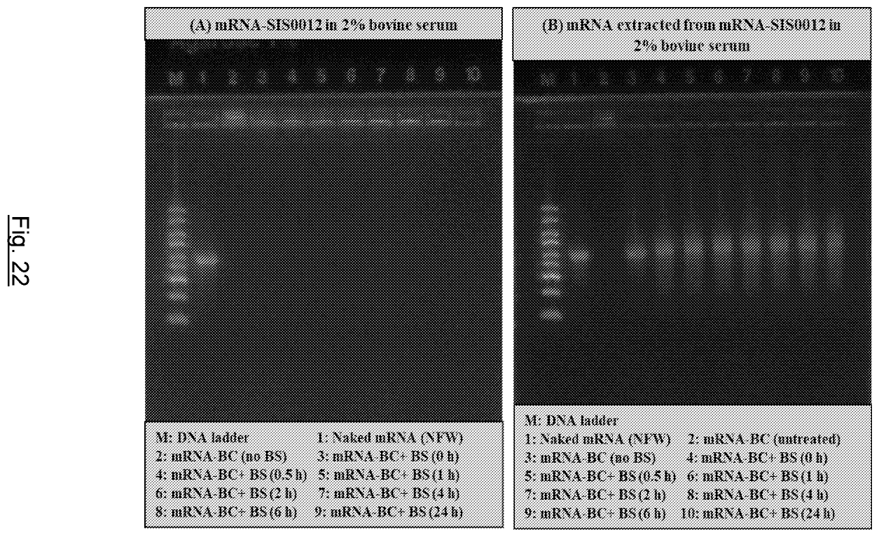

Aliquots were taken at 0 h, 0.5 h, 1 h, 2 h, 4 h, 6 h and 24 h for gel electrophoresis analysis (see FIGS. 22 to 25, discussed below).

For the purpose of the gel electrophoresis analysis, samples of mRNA extracted from SIS0012, SIS0013 and the lipid nanoparticle formulation, were also collected at each time point. To extract mRNA, samples were mixed with sodium dodecyl sulfate (1% solution in nuclease free water); mixed thoroughly by vortexing; and incubated at room temperature for 20 minutes. At the end of the incubation, KCl (0.1 M solution in nuclease free water) was added to the mixture, mixed well by vortexing and then incubated on ice for 10 minutes. The mixtures were then centrifuged at 4° C. at 19000×g (14224 rpm) for 15 minutes. The supernatant was carefully removed without disturbing the pellet and was transferred to a new PCR tube.

Gel electrophoresis analysis was performed using E-Gel™ Power Snap electrophoresis device and E-Gel™ agarose gels (1%), both available from ThermoFisher Scientific, 168 Third Avenue, Waltham, MA USA 02451.

### Discussion of Results

The gel electrophoresis results for mRNA-SIS0012, mRNA-SIS0013, mRNA-X (LNP without silicon) and naked mRNA, after incubation with 2% bovine serum at 0 h, 0.5 h, 1 h, 2 h, 4 h, 6 h and 24 h, are provided in FIGS. 22 to 25.

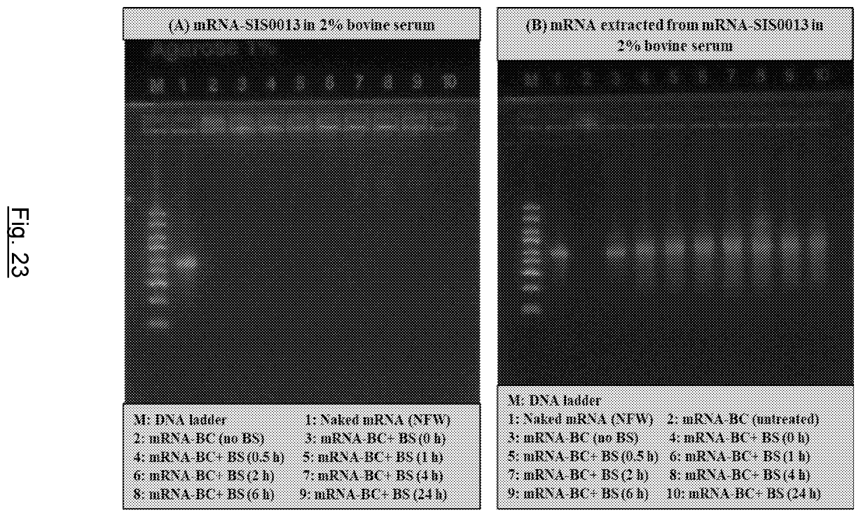

FIGS. 22A and 23A show that no mRNA migrated away from the mRNA-SIS0012 and mRNA-SIS0013 wells before or after incubation with serum, at any of the time points investigated.

Within the mRNA-SIS0012 and mRNA-SIS0013 wells, there was a strong mRNA signal for before any treatment with the serum; following exposure to serum, the signal within each of the mRNA-SIS0012 and mRNA-SIS0013 wells gradually reduced over time. Eventually (i.e., for the samples incubated for 6 h and 24 h) a relatively weak signal was observed in the mRNA-SIS0012 and mRNA-SIS0013 wells. The lack of mRNA migration away from the SIS0012 and SIS0013 wells indicates that SIS0012 and SIS0013 associate satisfactorily with mRNA. Over time in a serum environment at 37° C. (mimicking an in vivo environment to which mRNA may be exposed upon administration to a subject, wherein nuclease enzymes ae present) it is thought that the mRNA may gradually have degraded, resulting in a fainter in-well signal. Thus, the association of mRNA with SIS0012 and SIS0013 is satisfactory; and mRNA is protected from degradation over time, even in an in vivo-like environment, wherein nuclease enzymes are present, and which is held at a temperature of 37° C.

To confirm the above results, mRNA extracted from the mRNA-SIS0012 and mRNA-SIS0013 at all time points investigated, was subjected to gel electrophoresis analysis. This mRNA was observed to migrate through the gel, as shown in FIGS. 22B and 23B. There was a strong and sharp band for mRNA dissociated from SIS0012 and SIS0013 before incubation with serum (no serum control), which was as strong as the band observed for the naked mRNA control sample, and travelled the same distance, indicating the integrity of the SIS0012-bound mRNA and of the SIS0013-bound mRNA. In the case of the mRNA recovered from the mRNA-SIS0012 and mRNA-SIS0013 samples mixed with serum, there was a strong band for extracted mRNA for the time points from 0 h to 6 h; but after that, the strength of the band started to reduce over time and smearing was observed from 4 h onwards.

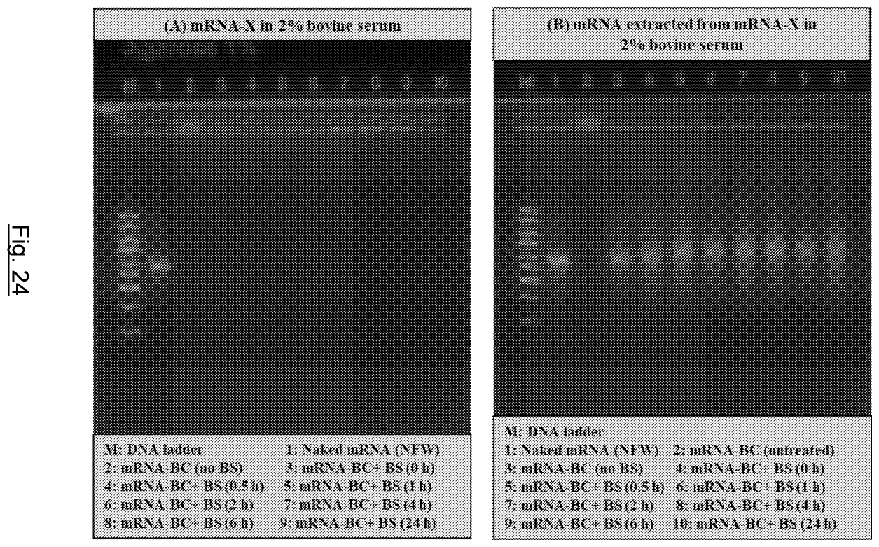

The gel electrophoresis results for the LNP counterpart formulated without silicon, dubbed mRNA-X are shown in FIG. 24. FIG. 24B (extracted mRNA) shows smearing that was more pronounced than SIS0012 and SIS0013. This indicates that LNP-associated mRNA is more susceptible to degradation upon exposure to serum, than SIS0012-associated or SIS0013-associated mRNA. The bands as early as 0.5 h for the LNP sample have a weak signal, indicating the presence of small fragments of mRNA that have travelled in the gel.

Thus, the successful complexation of mRNA with SIS0012 and SIS0013 was confirmed by gel retardation assay.

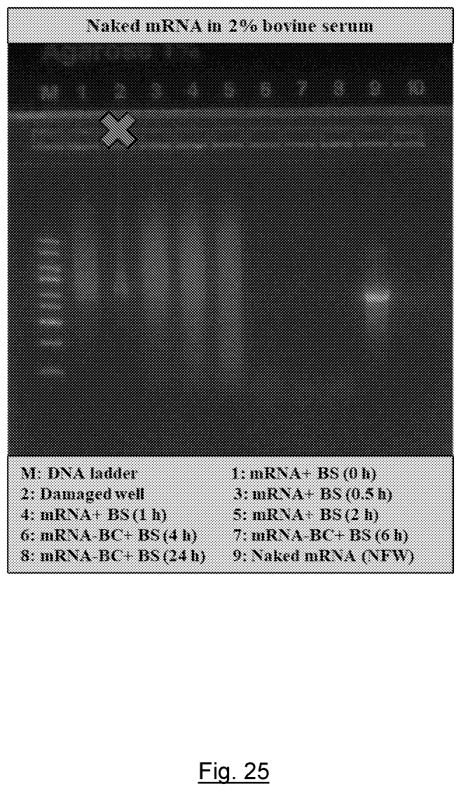

Summarising, these gel electrophoresis results demonstrate the protective action of SIS0012 and SIS0013 in respect of mRNA, against degradation by nuclease enzymes present in bovine serum. In contrast, in the case of mRNA associated with LNP without silicon (FIG. 24) the gel electrophoresis results show strong smearing of mRNA. As for naked mRNA, FIG. 25) shows fragmentation started shortly after incubation with serum (0.5 h) and degradation was observed almost immediately.

1. A nucleic acid vector composition comprising:
one or more enzymes and/or one or more fragments thereof;
a nucleic acid;
one or more particles comprising hydrolysable silicon; and
one or more lipids.

2. A nucleic acid vector composition according to claim 1, wherein the one or more enzymes comprise one or more enzymes for forming an enzyme-substrate complex with a nucleic acid.

3. A nucleic acid vector composition according to claim 1, wherein the one or more enzymes comprise one or more nucleases and/or one or more polymerases.

4. A nucleic acid vector composition according to claim 1, wherein the ratio of enzyme and/or fragment thereof to nucleic acid by weight is in the range of 1:1×10¹² to 1:1.

5. A nucleic acid vector composition according to claim 1, wherein the one or more enzymes comprise one or more enzymes having an activity, on a nucleic acid substrate, of at least 1 μmol min⁻¹ at a pH of 7.4 and a temperature of 25° C.

6. A nucleic acid vector composition according to claim 1, which is in the form of a lipid nanoparticle, optionally having a core encapsulated by a shell, wherein the shell comprises one or more lipids and the core comprises the nucleic acid and the one or more particles comprising hydrolysable silicon.

7. A nucleic acid vector according to claim 1, wherein the nucleic acid is mRNA, for example in vitro transcribed mRNA.

8. A nucleic acid vector according to claim 1, wherein the nucleic acid is DNA, for example plasmid DNA.

9. A nucleic acid vector composition according to claim 7, wherein the mRNA comprises a protein-encoding open reading frame and optionally one or more of:
a 5′ cap;
a poly (A) tail; and
one or more untranslated regions.

10. A nucleic acid vector composition according to claim 9, wherein the open reading frame of the mRNA encodes:
an antigen of a pathogen, optionally wherein the open reading frame of the mRNA encodes a spike protein antigen of SARS-CoV-2;
a tumour-associated antigen or a tumour-specific antigen;
an allergen; or
a modulator of an immune disease, an autoimmune disease, or an inflammatory disease.

11. A nucleic acid vector composition according to claim 1, further comprising an amino acid, and/or further comprising one or more disaccharides.

12. A method of preparing the nucleic acid vector composition according to claim 1, the method comprising:
obtaining the nucleic acid by in vitro transcription from a DNA template or purification from a biological source; and
combining the nucleic acid with the one or more particles and the one or more lipids.

13. A method of preparing a nucleic acid vector composition according to claim 12, wherein the nucleic acid is mRNA and wherein it is obtained by in vitro transcription from a DNA template.

14. A nucleic acid vector composition produced by the method according to claim 12.

15. A pharmaceutical composition comprising the nucleic acid vector composition according to claim 1, wherein the pharmaceutical composition is a prophylactic or therapeutic vaccine composition.

16. A pharmaceutical composition according to claim 15, wherein the nucleic acid has a half-life in the pharmaceutical composition at 4° C. of at least 3 months.

17. A pharmaceutical composition according to claim 15, wherein the nucleic acid has a half-life in the pharmaceutical composition at 4° C. of at least 6 months.

18. A pharmaceutical composition according to claim 15, wherein the pharmaceutical composition further comprises an adjuvant.

19. A pharmaceutical composition according to claim 15, wherein the pharmaceutical composition is in a form suitable for intramuscular injection.

20. (canceled)

21. A method of treating or preventing a disease or disorder, comprising: administering to a subject in need thereof a pharmaceutical composition according to claim 15.

22. A method of providing a vaccination to a subject, comprising oral, subcutaneous or intramuscular administration of a pharmaceutical composition according to claim 15.

23. (canceled)

24. (canceled)

25. (canceled)
